(19) United States
(12) Patent Application Publication
(10) Pub. No.: US 2025/0255685 A1
(43) Pub. Date: Aug. 14, 2025

(54) ROBOTIC SURGICAL SYSTEM WITH CONTEXT HAPTICS

(71) Applicant: MAKO Surgical Corp., Weston, FL (US)

(72) Inventors: David Bowling, Los Ranchos, NM (US); Jill Sarah Wolfson Jacobson, Weston, FL (US); Rishabh Khurana, Fort Lauderdale, FL (US)

(21) Appl. No.: 19/027,296

(22) Filed: Jan. 17, 2025

Related U.S. Application Data
(60) Provisional application No. 63/551,137, filed on Feb. 8, 2024.

(51) Int. Cl. A61B 34/37; A61B 34/00

(52) U.S. Cl. CPC A61B 34/37; A61B 34/76

(57) ABSTRACT

A method of controlling a robot of a surgical system includes monitoring positions of a first interaction point defined relative to a surgical instrument and as second interaction point defined relative to the surgical instrument, determining a first force feedback based on a first interaction between the first interaction and a boundary based on a first stiffness, determining a second force feedback based on a second interaction between the second interaction point and the boundary based on a second stiffness, and controlling the robot to provide a combined force feedback based on the first force feedback and the second force feedback.

### CROSS-REFERENCE TO RELATED APPLICATION

This application claims the benefit of and priority to U.S. Provisional Application No. 63/551,137, filed Feb. 8, 2024, the entire disclosure of which is incorporated by reference herein.

### BACKGROUND

The present disclosure relates generally to surgical systems for orthopedic surgeries, for example surgical systems that facilitate joint replacement procedures. Joint replacement procedures (arthroplasty procedures) are widely used to treat osteoarthritis and other damage to a patient's joint by replacing portions of the joint with prosthetic components. Joint replacement procedures can include procedures to replace hips, knees, shoulders, or other joints with one or more prosthetic components.

One possible tool for use in an arthroplasty procedure is a robotically-assisted surgical system. A robotically-assisted surgical system typically includes a robotic device that is used to prepare a patient's anatomy to receive an implant, a tracking system configured to monitor the location of the robotic device relative to the patient's anatomy, and a computing system configured to monitor and control the robotic device. Robotically-assisted surgical systems, in various forms, autonomously carry out surgical tasks, provide force feedback to a user manipulating a surgical device to complete surgical tasks, augment surgeon dexterity and precision, and/or provide other navigational cues to facilitate safe and accurate surgical operations.

A surgical plan is typically established prior to performing a surgical procedure with a robotically-assisted surgical system. Based on the surgical plan, the surgical system guides, controls, or limits movements of the surgical device during portions of the surgical procedure. Guidance and/or control of the surgical device serves to assist the surgeon during implementation of the surgical plan. Various features enhancing such guidance would be advantageous.

### BRIEF DESCRIPTION OF THE DRAWINGS

FIG. 1 is a perspective view of a femur prepared to receive an implant component, according to an exemplary embodiment.

FIG. 2 is an illustration of a surgical system, according to an exemplary embodiment.

FIG. 3 is a flowchart of a first process that can be executed by the surgical system of FIG. 2, according to an exemplary embodiment.

FIG. 4 is a flowchart of a second process that can be executed by the surgical system of FIG. 2, according to an exemplary embodiment.

FIG. 5 is an illustration of a robotic device, according to an exemplary embodiment.

FIG. 6 is a flowchart of a process for providing force feedback by a surgical system, according to some embodiments.

FIG. 7 is an illustration of interaction points and a boundary relating to the process of FIG. 6, according to some embodiments.

FIG. 8 is another illustration of interaction points and boundaries relating to the process of FIG. 6, according to some embodiments.

FIG. 9 is a flowchart of another process for providing force feedback by a surgical system, according to some embodiments.

FIG. 10 is an illustration of interaction points and boundaries relating to the process of FIG. 9, according to some embodiments.

FIG. 11 is a flowchart of another process for providing force feedback by a surgical system, according to some embodiments.

FIG. 12 is an illustration of interaction points and boundaries relating to the process of FIG. 11, according to some embodiments.

FIG. 13 is a flowchart of another process for providing force feedback by a surgical system, according to some embodiments.

FIG. 14 is a flowchart of another process for providing force feedback by a surgical system, according to some embodiments.

### SUMMARY

One implementation of the present disclosure is a method of operating a robot of a surgical system. The method includes monitoring positions of a first interaction point defined relative to a surgical instrument and as second interaction point defined relative to the surgical instrument, determining a first force feedback based on a first interaction between the first interaction and a boundary based on a first stiffness, determining a second force feedback based on a second interaction between the second interaction point and the boundary based on a second stiffness, and controlling the robot to provide a combined force feedback based on the first force feedback and the second force feedback.

Another implementation of the present disclosure is a method of operating a robot of a surgical system including monitoring positions of a first interaction point defined relative to a surgical instrument and as second interaction point defined relative to the surgical instrument, determining a first force feedback based on a first interaction between the first interaction and a first boundary based on a first stiffness, determining a second force feedback based on a second interaction between the second interaction point and a second boundary based on a second stiffness, and controlling the robot to provide a combined force feedback based on the first force feedback and the second force feedback.

Another implementation of the present disclosure is a method of operating a robot of a surgical system, including monitoring a position of a first interaction point defined relative to a surgical instrument, controlling the robot to provide a force feedback on the surgical instrument based on interaction between the first interaction point and a haptic boundary with the force feedback being based on a stiffness of the haptic boundary, and reducing the stiffness of the haptic boundary in response to occurrence of a threshold amount of under-resection relative to a planned resection.

### DETAILED DESCRIPTION

Presently preferred embodiments of the invention are illustrated in the drawings. An effort has been made to use the same or like reference numbers throughout the drawings to refer to the same or like parts. Although this specification refers primarily to a robotic arm for orthopedic joint replacement, it should be understood that the subject matter described herein is applicable to other types of robotic systems, including those used for non-surgical applications, as well as for procedures directed to other anatomical regions, for example spinal or dental procedures.

### Surgical Robotics System

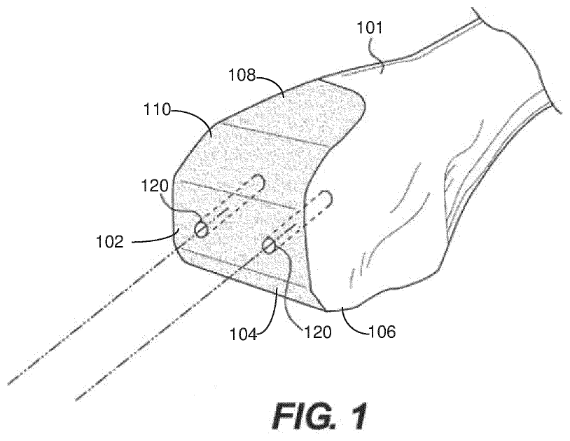

Referring now to FIG. 1, a femur 101 as modified during a knee arthroplasty procedure is shown, according to an exemplary embodiment. As shown in FIG. 1, the femur 101 has been modified with multiple planar cuts. In the example shown, the femur 101 has been modified by five substantially planar cuts to create five substantially planar surfaces, namely distal surface 102, posterior chamfer surface 104, posterior surface 106, anterior surface 108, and anterior chamfer surface 110. The planar surfaces may be achieved using a sagittal saw or other surgical device, for example a surgical device coupled to a robotic device as in the examples described below. The planar surfaces 102-110 are created such that the planar surfaces 102-110 will mate with corresponding surfaces of a femoral implant component. The positions and angular orientations of the planar surfaces 102-110 may determine the alignment and positioning of the implant component. Accordingly, operating a surgical device to create the planar surfaces 102-110 with a high degree of accuracy may improve the outcome of a joint replacement procedure.

As shown in FIG. 1, the femur 101 has also been modified to have a pair of pilot holes 120. The pilot holes 120 extend into the femur 101 and are created such that the pilot holes 120 can receive a screw, a projection extending from a surface of an implant component, or other structure configured to facilitate coupling of an implant component to the femur 101. The pilot holes 120 may be created using a drill, spherical burr, or other surgical device as described herein. The pilot holes 120 may have a pre-planned position, orientation, and depth, which facilitates secure coupling of the implant component to the bone in a desired position and orientation. In some cases, the pilot holes 120 are planned to intersect with higher-density areas of a bone and/or to avoid other implant components and/or sensitive anatomical features. Accordingly, operating a surgical device to create the pilot holes 120 with a high degree of accuracy may improve the outcome of a joint replacement procedure.

A tibia may also be modified during a joint replacement procedure. For example, a planar surface may be created on the tibia at the knee joint to prepare the tibia to mate with a tibial implant component. In some embodiments, one or more pilot holes 120 or other recess (e.g., fin-shaped recess) may also be created in the tibia to facilitate secure coupling of an implant component tot eh bone.

In some embodiments, the systems and methods described herein provide robotic assistance for creating the planar surfaces 102-110 and the pilot holes 120 at the femur 101, and/or a planar surface and/or pilot holes 120 or other recess on a tibia. It should be understood that the creation of five planar cuts and two cylindrical pilot holes as shown in FIG. 1 is an example only, and that the systems and methods described herein may be adapted to plan and facilitate creation of any number of planar or non-planar cuts, any number of pilot holes, any combination thereof, etc., for preparation of any bone and/or joint in various embodiments. For example, in a hip or shoulder arthroplasty procedure, a spherical burr may be used in accordance with the systems and methods herein to ream a curved surface configured to receive a curved implant cup. Furthermore, in other embodiments, the systems and methods described herein may be used to facilitate placement an implant component relative to a bone (e.g., to facilitate impaction of cup implant in a hip arthroplasty procedure). Many such surgical and non-surgical implementations are within the scope of the present disclosure.

The positions and orientations of the planar surfaces 102-110, pilot holes 120, and any other surfaces or recesses created on bones of the knee joint can affect how well implant components mate to the bone as well as the resulting biomechanics for the patient after completion of the surgery. Tension on soft tissue can also be affected. Accordingly, systems and methods for planning the cuts which create these surfaces, facilitating intra-operative adjustments to the surgical plan, and providing robotic-assistance or other guidance for facilitating accurate creation of the planar surfaces 102-110, other surfaces, pilot holes 120, or other recesses can make surgical procedures easier and more efficient for healthcare providers and improve surgical outcomes.

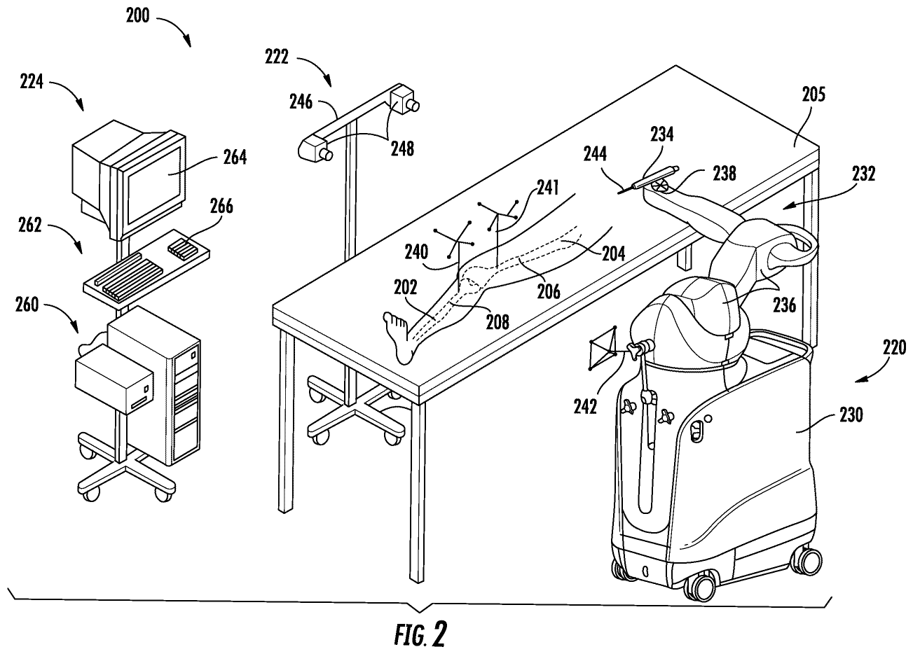

Referring now to FIG. 2, a surgical system 200 for orthopedic surgery is shown, according to an exemplary embodiment. In general, the surgical system 200 is configured to facilitate the planning and execution of a surgical plan, for example to facilitate a joint-related procedure. As shown in FIG. 2, the surgical system 200 is set up to treat a leg 202 of a patient 204 sitting or lying on table 205. In the illustration shown in FIG. 2, the leg 202 includes femur 206 (e.g., femur 101 of FIG. 1) and tibia 208, between which a prosthetic knee implant is to be implanted in a total knee arthroscopy procedure. In other scenarios, the surgical system 200 is set up to treat a hip of a patient, e.g., the femur and the pelvis of the patient. Additionally, in still other scenarios, the surgical system 200 is set up to treat a shoulder of a patient, e.g., to facilitate replacement and/or augmentation of components of a shoulder joint (e.g., to facilitate placement of a humeral component, a glenoid component, and a graft or implant augment). Various other anatomical regions and procedures are also possible.

The robotic device 220 is configured to modify a patient's anatomy (e.g., femur 206 of patient 204) under the control of the computing system 224. One embodiment of the robotic device 220 is a haptic device. “Haptic” refers to a sense of touch, and the field of haptics relates to, among other things, human interactive devices that provide feedback to an operator. Feedback may include tactile sensations such as, for example, vibration. Feedback may also include providing force to a user, such as a positive force or a resistance to movement. One use of haptics is to provide a user of the device with guidance or limits for manipulation of that device. For example, a haptic device may be coupled to a surgical device, which can be manipulated by a surgeon to perform a surgical procedure. The surgeon's manipulation of the surgical device can be guided or limited through the use of haptics to provide feedback to the surgeon during manipulation of the surgical device.

Another embodiment of the robotic device 220 is an autonomous or semi-autonomous robot. “Autonomous” refers to a robotic device's ability to act independently or semi-independently of human control by gathering information about its situation, determining a course of action, and automatically carrying out that course of action. For example, in such an embodiment, the robotic device 220, in communication with the tracking system 222 and the computing system 224, may autonomously complete the series of femoral cuts mentioned above without direct human intervention.

The robotic device 220 includes a base 230, a robotic arm 232, and a surgical device 234, and is communicably coupled to the computing system 224 and the tracking system 222. The base 230 provides a moveable foundation for the robotic arm 232, allowing the robotic arm 232 and the surgical device 234 to be repositioned as needed relative to the patient 204 and the table 205. The base 230 may also contain power systems, computing elements, motors, and other electronic or mechanical system necessary for the functions of the robotic arm 232 and the surgical device 234 described below.

The robotic arm (robot) 232 is configured to support the surgical device 234 and provide a force as instructed by the computing system 224. In some embodiments, the robotic arm 232 allows a user to manipulate the surgical device and provides force feedback to the user. In such an embodiment, the robotic arm 232 includes joints 236 and mount 238 that include motors, actuators, or other mechanisms configured to allow a user to freely translate and rotate the robotic arm 232 and surgical device 234 through allowable poses while providing force feedback to constrain or prevent some movements of the robotic arm 232 and surgical device 234 as instructed by computing system 224. As described in detail below according to various embodiments, the robotic arm 232 thereby allows a surgeon to have full control over the surgical device 234 within a control object while providing force feedback along a boundary of that object (e.g., a vibration, a force preventing or resisting penetration of the boundary). In some embodiments, the robotic arm 232 is configured to move the surgical device to a new pose automatically without direct user manipulation, as instructed by computing system 224, in order to position the robotic arm 232 as needed and/or complete certain surgical tasks, including, for example, cuts in a femur 206.

The surgical device 234 is configured to cut, burr, grind, drill, partially resect, reshape, and/or otherwise modify a bone. The surgical device 234 may be any suitable tool, and may be one of multiple tools interchangeably connectable to robotic device 220. For example, as shown in FIG. 2 the surgical device 234 includes a spherical burr 244. In other examples, the surgical device 234 may also be a sagittal saw, for example with a blade aligned parallel with a tool axis or perpendicular to the tool axis. The surgical device 234 may also be a drill, for example with a rotary bit aligned parallel with a tool axis or perpendicular to the tool axis. The surgical device 234 may also be a holding arm or other support configured to hold an implant component (e.g., cup, implant augment, etc.) in position while the implant component is screwed to a bone, adhered (e.g., cemented) to a bone or other implant component, or otherwise installed in a preferred position. In some embodiments, the surgical device 234 is an impaction tool configured to provide an impaction force to a cup implant to facilitate fixation of the cup implant to a pelvis in a planned location and orientation.

Tracking system 222 is configured track the patient's anatomy (e.g., femur 206 and tibia 208) and the robotic device 220 (e.g., surgical device 234 and/or robotic arm 232) to enable control of the surgical device 234 coupled to the robotic arm 232, to determine a position and orientation of modifications or other results made by the surgical device 234, and allow a user to visualize the bones (e.g., femur 206, the tibia 208, pelvis, humerus, scapula, etc. as applicable in various procedures), the surgical device 234, and/or the robotic arm 232 on a display of the computing system 224. The tracking system 222 can also be used to collect biomechanical measurements relating to the patient's anatomy, assess joint gap distances, identify a hip center point, assess native or corrected joint deformities, or otherwise collect information relating to the relative poses of anatomical features. More particularly, the tracking system 222 determines a position and orientation (e.g., pose) of objects (e.g., surgical device 234, femur 206) with respect to a coordinate frame of reference and tracks (e.g., continuously determines) the pose of the objects during a surgical procedure. According to various embodiments, the tracking system 222 may be any type of navigation system, including a non-mechanical tracking system (e.g., an optical tracking system), a mechanical tracking system (e.g., tracking based on measuring the relative angles of joints 236 of the robotic arm 232), or any combination of non-mechanical and mechanical tracking systems.

In the embodiment shown in FIG. 2, the tracking system 222 includes an optical tracking system. Accordingly, tracking system 222 includes a first fiducial tree 240 coupled to the tibia 208, a second fiducial tree 241 coupled to the femur 206, a third fiducial tree 242 coupled to the base 230, one or more fiducials attachable to surgical device 234, and a detection device 246 configured to detect the three-dimensional position of fiducials (e.g., markers on fiducial trees 240-242). Fiducial trees 240, 241 may be coupled to other bones as suitable for various procedures (e.g., pelvis and femur in a hip arthroplasty procedure). Detection device 246 may be an optical detector such as a camera or infrared sensor. The fiducial trees 240-242 include fiducials, which are markers configured to show up clearly to the optical detector and/or be easily detectable by an image processing system using data from the optical detector, for example by being highly reflective of infrared radiation (e.g., emitted by an element of tracking system 222). In some embodiments, the markers are active light emitting diodes. A stereoscopic arrangement of cameras 248 on detection device 246 allows the position of each fiducial to be determined in 3D-space through a triangulation approach in the example shown. Each fiducial has a geometric relationship to a corresponding object, such that tracking of the fiducials allows for the tracking of the object (e.g., tracking the second fiducial tree 241 allows the tracking system 222 to track the femur 206), and the tracking system 222 may be configured to carry out a registration process to determine or verify this geometric relationship. Unique arrangements of the fiducials in the fiducial trees 240-242 (e.g., the fiducials in the first fiducial tree 240 are arranged in a different geometry than fiducials in the second fiducial tree 241) allows for distinguishing the fiducial trees, and therefore the objects being tracked, from one another.

Using the tracking system 222 of FIG. 2 or some other approach to surgical navigation and tracking, the surgical system 200 can determine the position of the surgical device 234 relative to a patient's anatomical feature, for example femur 206, as the surgical device 234 is used to modify the anatomical feature or otherwise facilitate the surgical procedure. Additionally, using the tracking system 222 of FIG. 2 or some other approach to surgical navigation and tracking, the surgical system 200 can determine the relative poses of the tracked bones.

The computing system 224 is configured to create a surgical plan, control the robotic device 220 in accordance with the surgical plan to make one or more bone modifications and/or facilitate implantation of one or more prosthetic components. Accordingly, the computing system 224 is communicably coupled to the tracking system 222 and the robotic device 220 to facilitate electronic communication between the robotic device 220, the tracking system 222, and the computing system 224. Further, the computing system 224 may be connected to a network to receive information related to a patient's medical history or other patient profile information, medical imaging, surgical plans, surgical procedures, and to perform various functions related to performance of surgical procedures, for example by accessing an electronic health records system. Computing system 224 includes processing circuit 260 and input/output device 262. Computing system 224 may include circuitry configured to enable the operations described herein, for example using processing circuit 260 and/or input/output device 262.

The input/output device 262 is configured to receive user input and display output as needed for the functions and processes described herein. As shown in FIG. 2, input/output device 262 includes a display 264 and a keyboard 266. The display 264 is configured to display graphical user interfaces generated by the processing circuit 260 that include, for example, information about surgical plans, medical imaging, settings and other options for surgical system 200, status information relating to the tracking system 222 and the robotic device 220, and tracking visualizations based on data supplied by tracking system 222. The keyboard 266 is configured to receive user input to those graphical user interfaces to control one or more functions of the surgical system 200.

The processing circuit 260 includes a processor and memory device. The processor can be implemented as a general purpose processor, an application specific integrated circuit (ASIC), one or more field programmable gate arrays (FPGAs), a group of processing components, or other suitable electronic processing components. The memory device (e.g., memory, memory unit, storage device, etc.) is one or more devices (e.g., RAM, ROM, Flash memory, hard disk storage, etc.) for storing data and/or computer-readable media for completing or facilitating the various processes and functions described in the present application. The memory device may be or include volatile memory or non-volatile memory. The memory device may include database components, object code components, script components, or any other type of information structure for supporting the various activities and information structures described in the present application. According to an exemplary embodiment, the memory device is communicably connected to the processor via the processing circuit 260 and includes computer-readable media for executing (e.g., by the processing circuit 260 and/or processor) one or more processes described herein, for example non-transitory computer-readable media.

More particularly, processing circuit 260 is configured to facilitate the creation of a preoperative surgical plan prior to the surgical procedure. According to some embodiments, the preoperative surgical plan is developed utilizing a three-dimensional representation of a patient's anatomy, also referred to herein as a “virtual bone model.” A “virtual bone model” may include virtual representations of cartilage or other tissue in addition to bone. To obtain the virtual bone model, the processing circuit 260 receives imaging data of the patient's anatomy on which the surgical procedure is to be performed. The imaging data may be created using any suitable medical imaging technique to image the relevant anatomical feature, including computed tomography (CT), magnetic resonance imaging (MRI), and/or ultrasound. The imaging data is then segmented (e.g., the regions in the imaging corresponding to different anatomical features are distinguished) to obtain the virtual bone model. For example, MRI-based scan data of a joint can be segmented to distinguish bone from surrounding ligaments, cartilage, previously-implanted prosthetic components, and other tissue to obtain a three-dimensional model of the imaged bone.

Alternatively, the virtual bone model may be obtained by selecting a three-dimensional model from a database or library of bone models. In one embodiment, the user may use input/output device 262 to select an appropriate model. In another embodiment, the processing circuit 260 may execute stored instructions to select an appropriate model based on images or other information provided about the patient. The selected bone model(s) from the database can then be deformed based on specific patient characteristics, creating a virtual bone model for use in surgical planning and implementation as described herein.

A preoperative surgical plan can then be created based on the virtual bone model. The surgical plan may be automatically generated by the processing circuit 260, input by a user via input/output device 262, or some combination of the two (e.g., the processing circuit 260 limits some features of user-created plans, generates a plan that a user can modify, etc.). In some embodiments, the surgical plan may be generated and/or modified based on distraction force measurements collected intraoperatively.

The preoperative surgical plan includes the desired cuts, holes, surfaces, burrs, or other modifications to a patient's anatomy to be made using the surgical system 200. For example, for a total knee arthroscopy procedure, the preoperative plan may include the cuts necessary to form, on a femur, a distal surface, a posterior chamfer surface, a posterior surface, an anterior surface, and an anterior chamfer surface in relative orientations and positions suitable to be mated to corresponding surfaces of the prosthetic to be joined to the femur during the surgical procedure, as well as cuts necessary to form, on the tibia, surface(s) suitable to mate to the prosthetic to be joined to the tibia during the surgical procedure. As another example, the preoperative plan may include the modifications necessary to create holes (e.g., pilot holes 120) in a bone. As another example, in a hip arthroplasty procedure, the surgical plan may include the burr necessary to form one or more surfaces on the acetabular region of the pelvis to receive a cup and, in suitable cases, an implant augment. Accordingly, the processing circuit 260 may receive, access, and/or store a model of the prosthetic to facilitate the generation of surgical plans. In some embodiments, the processing circuit facilitate intraoperative modifications to the preoperative plant.

The processing circuit 260 is further configured to generate a control object for the robotic device 220 in accordance with the surgical plan. The control object may take various forms according to the various types of possible robotic devices (e.g., haptic, autonomous). For example, in some embodiments, the control object defines instructions for the robotic device 220 to control the robotic device 220 to move within the control object (e.g., to autonomously make one or more cuts of the surgical plan guided by feedback from the tracking system 222). In some embodiments, the control object includes a visualization of the surgical plan and the robotic device 220 on the display 264 to facilitate surgical navigation and help guide a surgeon to follow the surgical plan (e.g., without active control or force feedback of the robotic device). In embodiments where the robotic device 220 is a haptic device, the control object may be a haptic object as described in the following paragraphs.

In an embodiment where the robotic device 220 is a haptic device, the processing circuit 260 is further configured to generate one or more haptic objects based on the preoperative surgical plan to assist the surgeon during implementation of the surgical plan by enabling constraint of the surgical device 234 during the surgical procedure. A haptic object may be formed in one, two, or three dimensions. For example, a haptic object can be a line, a plane, or a three-dimensional volume. A haptic object may be curved with curved surfaces and/or have flat surfaces, and can be any shape, for example a funnel shape. Haptic objects can be created to represent a variety of desired outcomes for movement of the surgical device 234 during the surgical procedure. One or more of the boundaries of a three-dimensional haptic object may represent one or more modifications, such as cuts, to be created on the surface of a bone. A planar haptic object may represent a modification, such as a cut, to be created on the surface of a bone. A curved haptic object may represent a resulting surface of a bone as modified to receive a cup implant and/or implant augment. A line haptic object may correspond to a pilot hole to be made in a bone to prepare the bone to receive a screw or other projection.

In an embodiment where the robotic device 220 is a haptic device, the processing circuit 260 is further configured to generate a virtual tool representation of the surgical device 234. The virtual tool includes one or more haptic interaction points (HIPs), which represent and are associated with locations on the surgical device 234. In an embodiment in which the surgical device 234 is a spherical burr (e.g., as shown in FIG. 2), a HIP may represent the center of the spherical burr. Where one HIP is used to virtually represent a surgical device, the HIP may be referred to herein as a tool center point (TCP). If the surgical device 234 is an irregular shape, for example as for a sagittal saw, the virtual representation of the sagittal saw may include numerous HIPs. Using multiple HIPs to generate haptic forces (e.g. positive force feedback or resistance to movement) on a surgical device is described in U.S. application Ser. No. 13/339,369, titled “System and Method for Providing Substantially Stable Haptics,” filed Dec. 28, 2011, and hereby incorporated by reference herein in its entirety. In one embodiment of the present invention, a virtual tool representing a sagittal saw includes eleven HIPs. As used herein, references to an “HIP” are deemed to also include references to “one or more HIPs.” As described below, relationships between HIPs and haptic objects enable the surgical system 200 to constrain the surgical device 234.

Prior to performance of the surgical procedure, the patient's anatomy (e.g., femur 206) is registered to the virtual bone model of the patient's anatomy by any known registration technique. One possible registration technique is point-based registration, as described in U.S. Pat. No. 8,010,180, titled “Haptic Guidance System and Method,” granted Aug. 30, 2011, and hereby incorporated by reference herein in its entirety. Alternatively, registration may be accomplished by 2D/3D registration utilizing a hand-held radiographic imaging device, as described in U.S. application Ser. No. 13/562,163, titled “Radiographic Imaging Device,” filed Jul. 30, 2012, and hereby incorporated by reference herein in its entirety. Registration also includes registration of the surgical device 234 to a virtual tool representation of the surgical device 234, so that the surgical system 200 can determine and monitor the pose of the surgical device 234 relative to the patient (e.g., to femur 206). Registration of allows for accurate navigation, control, and/or force feedback during the surgical procedure.

The processing circuit 260 is configured to monitor the virtual positions of the virtual tool representation, the virtual bone model, and the control object (e.g., virtual haptic objects) corresponding to the real-world positions of the patient's bone (e.g., femur 206), the surgical device 234, and one or more lines, planes, or three-dimensional spaces defined by forces created by robotic device 220. For example, if the patient's anatomy moves during the surgical procedure as tracked by the tracking system 222, the processing circuit 260 correspondingly moves the virtual bone model. The virtual bone model therefore corresponds to, or is associated with, the patient's actual (i.e. physical) anatomy and the position and orientation of that anatomy in real/physical space. Similarly, any haptic objects, control objects, or other planned automated robotic device motions created during surgical planning that are linked to cuts, modifications, etc. to be made to that anatomy also move in correspondence with the patient's anatomy. In some embodiments, the surgical system 200 includes a clamp or brace to substantially immobilize the femur 206 to minimize the need to track and process motion of the femur 206.

For embodiments where the robotic device 220 is a haptic device, the surgical system 200 is configured to constrain the surgical device 234 based on relationships between HIPs and haptic objects. That is, when the processing circuit 260 uses data supplied by tracking system 222 to detect that a user is manipulating the surgical device 234 to bring a HIP in virtual contact with a haptic object, the processing circuit 260 generates a control signal to the robotic arm 232 to provide haptic feedback (e.g., a force, a vibration) to the user to communicate a constraint on the movement of the surgical device 234. In general, the term “constrain,” as used herein, is used to describe a tendency to restrict movement. However, the form of constraint imposed on surgical device 234 depends on the form of the relevant haptic object. A haptic object may be formed in any desirable shape or configuration. As noted above, three exemplary embodiments include a line, plane, or three-dimensional volume. In one embodiment, the surgical device 234 is constrained because a HIP of surgical device 234 is restricted to movement along a linear haptic object. In another embodiment, the haptic object is a three-dimensional volume and the surgical device 234 may be constrained by substantially preventing movement of the HIP outside of the volume enclosed by the walls of the three-dimensional haptic object. In another embodiment, the surgical device 234 is constrained because a planar haptic object substantially prevents movement of the HIP outside of the plane and outside of the boundaries of the planar haptic object. For example, the processing circuit 260 can establish a planar haptic object corresponding to a planned planar distal cut needed to create a distal surface on the femur 206 in order to confine the surgical device 234 substantially to the plane needed to carry out the planned distal cut.

For embodiments where the robotic device 220 is an autonomous device, the surgical system 200 is configured to autonomously move and operate the surgical device 234 in accordance with the control object. For example, the control object may define areas relative to the femur 206 for which a cut should be made. In such a case, one or more motors, actuators, and/or other mechanisms of the robotic arm 232 and the surgical device 234 are controllable to cause the surgical device 234 to move and operate as necessary within the control object to make a planned cut, for example using tracking data from the tracking system 222 to allow for closed-loop control.

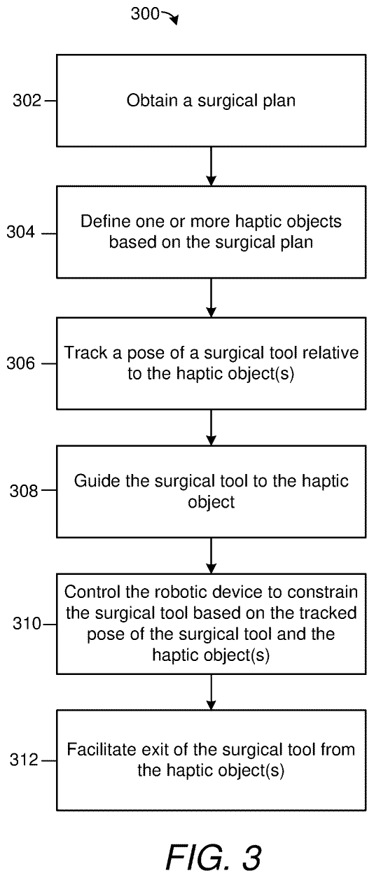

Referring now to FIG. 3, a flowchart of a process 300 that can be executed by the surgical system 200 of FIG. 2 is shown, according to an exemplary embodiment. Process 300 may be adapted to facilitate various surgical procedures, including total and partial joint replacement surgeries.

At step 302, a surgical plan is obtained. The surgical plan (e.g., a computer-readable data file) may define a desired outcome of bone modifications, for example defined based on a desired position of prosthetic components relative to the patient's anatomy. For example, in the case of a knee arthroplasty procedure, the surgical plan may provide planned positions and orientations of the planar surfaces 102-110 and the pilot holes 120 as shown in FIG. 1. The surgical plan may be generated based on medical imaging, 3D modeling, surgeon input, etc.

At step 304, one or more control boundaries, such as haptic objects, are defined based on the surgical plan. The one or more haptic objects may be one-dimensional (e.g., a line haptic), two dimensional (e.g., planar), or three dimensional (e.g., cylindrical, funnel-shaped, curved, etc.). The haptic objects may represent planned bone modifications (e.g., a haptic object for each of the planar surfaces 102-110 and each of the pilot holes 120 shown in FIG. 1), implant components, surgical approach trajectories, etc. defined by the surgical plan. The haptic objects can be oriented and positioned in three-dimensional space relative to a tracked position of a patient's anatomy.

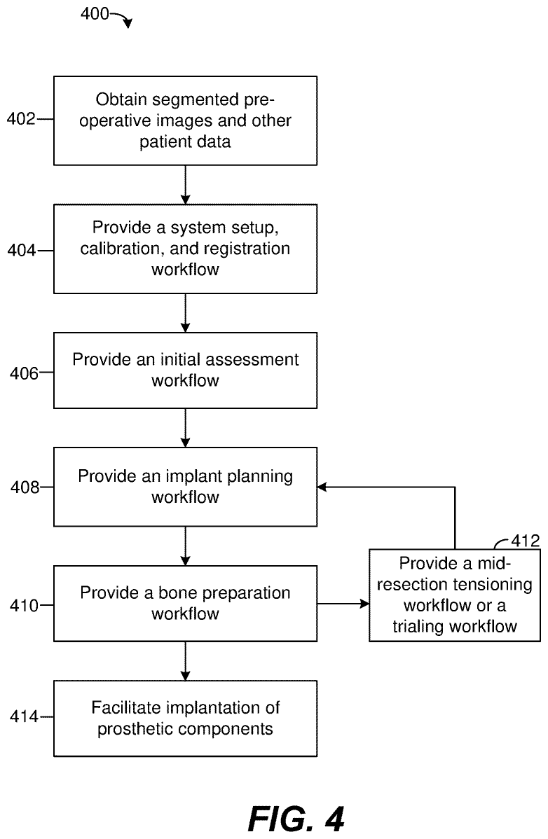

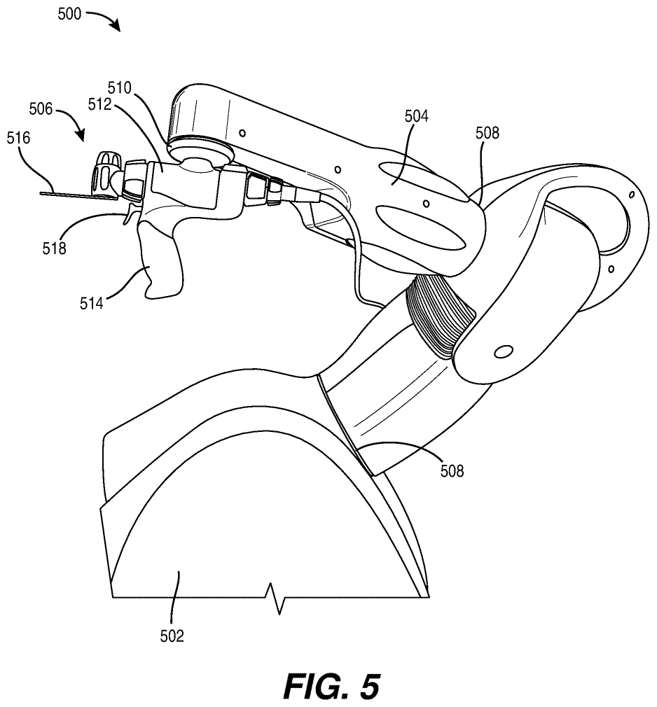

At step 306, a pose of a surgical device is tracked relative to the haptic object(s), for example by the tracking system 222 described above. In some embodiments, one point on the surgical device is tracked. In other embodiments, (e.g., in the example of FIGS. 4-5) two points on the surgical device are tracked, for example a tool center point (TCP) at a tip/effective end of the surgical device and a second interaction point (SIP) positioned along a body or handle portion of the surgical device. In other embodiments, three or more points on the surgical device are tracked. A pose of the surgical device is ascertained relative to a coordinate system in which the one or more haptic objects are defined and, in some embodiments, in which the pose of one or more anatomical features of the patient is also tracked.

At step 308, the surgical device is guided to the haptic object(s). For example, the display 264 of the surgical system 200 may display a graphical user interface instructing a user on how (e.g., which direction) to move the surgical device and/or robotic device to bring the surgical device to a haptic object. As another example, the surgical device may be guided to a haptic object using a collapsing haptic boundary as described in U.S. Pat. No. 9,289,264, the entire disclosure of which is incorporated by reference herein. As another example, the robotic device may be controlled to automatically move the surgical device to a haptic object.

In an embodiment where the robotic device is controlled to automatically move the surgical device to the haptic object (referred to as motorized alignment or automated alignment), the robotic device may be controlled so that a duration of the alignment is bounded by preset upper and lower time thresholds. That is, across various instances of process 300 and multiple procedures, automated alignment in step 308 may be configured to always take between a first amount of time (the lower time threshold) and a second amount of time (the upper time threshold). The lower time threshold may be selected such that the robotic device moves over a long enough duration to be perceived as well-controlled and to minimize collision or other risks associated with high speed. The upper time threshold may be selected such that the robotic device moves over a short enough duration to avoid user impatience and provide improved usability. For example, the upper time threshold hold may be approximately five seconds in an example where the lower time thresholds is approximately three seconds. In other embodiments, a single duration setpoint is used (e.g., four seconds). Step 308 can include optimizing a path for the robotic device such that the step 308 ensures successful alignment to the haptic object while also satisfying the upper and lower time thresholds or duration setpoint.

At step 310, the robotic device is controlled to constrain movement of the surgical device based on the tracked pose of the surgical device and the poses of one or more haptic objects. The constraining of the surgical device may be achieved as described above with reference to FIG. 2.

At step 312, exit of the surgical device from the haptic object(s) is facilitated, e.g., to release the constraints of a haptic object. For example, in some embodiments, the robotic device is controlled to allow the surgical device to exit a haptic object along an axis of the haptic object. In some embodiments, the surgical device may be allowed to exit the haptic object in a pre-determined direction relative to the haptic object. The surgical device may thereby be removed from the surgical field and the haptic object to facilitate subsequent steps of the surgical procedure. Additionally, it should be understood that, in some cases, the process 300 may return to step 308 where the surgical device is guided to the same or different haptic object after exiting a haptic object at step 312.

Process 300 may thereby be executed by the surgical system 200 to facilitate a surgical procedure. Features of process 300 are shown in FIGS. 4-14 below according to some embodiments, and such features can be combined in various combinations in various embodiments and/or based on settings selected for a particular procedure. Furthermore, it should be understood that the features of FIGS. 4-14 may be provided while omitting some or all other steps of process 300. All such possibilities are within the scope of the present disclosure.

Referring now to FIG. 4, a flowchart of a process 400 for facilitating surgical planning and guidance is shown, according to an exemplary embodiment. The process 400 may be executed by the surgical system 200 of FIG. 2, in some embodiments. In some cases, the process 300 is executed as part of executing the process 400.

At step 402, segmented pre-operative images and other patient data are obtained, for example by the surgical system 200. For example, segmented pre-operative CT images or MRI images may be received at the computing system 224 from an external server. In some cases, pre-operative images of a patient's anatomy are collected using an imaging device and segmented by a separate computing system and/or with manual user input to facilitate segmentation. In other embodiments, unsegmented pre-operative images are received at the computing system 224 and the computing system 224 is configured to automatically segment the images. The segmented pre-operative images can show the geometry, shape, size, density, and/or other characteristics of bones of a joint which is to be operated on in a procedure performed using process 400.

Other patient data can also be obtained at step 402. For example, the computing system 224 may receive patient information from an electronic medical records system. As another example, the computing system 224 may accept user input of patient information. The other patient data may include a patient's name, identification number, biographical information (e.g., age, weight, etc.), other health conditions, etc. In some embodiments, the patient data obtained at step 402 includes information specific to the procedure to be performed and the relevant pre-operative diagnosis. For example, the patient data may indicate which joint the procedure will be performed on (e.g., right knee, left knee). The patient data may indicate a diagnosed deformity, for example indicating whether a knee joint was diagnosed as having a varus deformity or a valgus deformity. This or other data that may facilitate the surgical procedure may be obtained at step 402.

At step 404, a system setup, calibration, and registration workflow is provided, for example by the surgical system 200. The system setup, calibration, and registration workflows may be configured to prepare the surgical system 200 for use in facilitating a surgical procedure. For example, at step 404, the computing system 224 may operate to provide graphical user interfaces that include instructions for performing system setup, calibration, and registrations steps. The computing system 224 may also cause the tracking system 222 to collect tracking data and control the robotic device 220 to facilitate system setup, calibration, and/or registration. The computing system 224 may also receiving tracking data from the tracking system 222 and information from the computing system 224 and use the received information and data to calibrate the robotic device 220 and define various geometric relationships between tracked points (e.g., fiducials, markers), other components of the surgical system 200 (e.g., robotic arm 232, surgical device 234, probe), and virtual representations of anatomical features (e.g., virtual bone models).

The system setup workflow provided at step 404 may include guiding the robotic device 220 to a position relative to a surgical table and the patient which will be suitable for completing an entire surgical procedure without repositioning the robotic device 220. For example, the computing system 224 may generate and provide a graphical user interface configured to provide instructions for moving a portable cart of the robotic device 220 into a preferred position. In some embodiments, the robotic device 220 can be tracked to determine whether the robotic device 220 is properly positioned. Once the cart is positioned, in some embodiments the robotic device 220 is controlled to automatically position the robotic arm 232 in a pose suitable for initiation of calibration and/or registration workflows.

The calibration and registration workflows provided at step 404 may include generating instructions for a user to perform various calibration and registration tasks while operating the tracking system 222 to generate tracking data. The tracking data can then be used to calibrate the tracking system 222 and the robotic device 220 and to register the first fiducial tree 240, second fiducial tree 241, and third fiducial tree 242 relative to the patient's anatomical features, for example by defining geometric relationships between the fiducial trees 240-242 and relevant bones of the patient in the example of FIG. 2. The registration workflow may include tracking a probe used to touch various points on the bones of a joint. In some embodiments, providing the registration workflow may include providing instructions to couple a checkpoint (e.g., a screw or pin configured to be contacted by a probe) to a bone and tracking a probe as the probe contacts the checkpoint and as the probe is used to paint (e.g., move along, touch many points along) one or more surfaces of the bone. The probe can be moved and tracked in order to collect points in or proximate the joint to be operated upon as well as at other points on the bone (e.g., at ankle or hip for a knee surgery).

In some embodiments, providing the registration workflow includes generating instructions to move the patient's leg to facilitate collection of relevant tracking data that can be used to identify the location of a biomechanical feature, for example a hip center point. Providing the registration workflow can include providing audio or visual feedback indicating whether the leg was moved in the proper manner to collect sufficient tracking data. Various methods and approaches for registration and calibration can be used in various embodiments. Step 404 may include steps performed before or after an initial surgical incision is made in the patient's skin to initiate the surgical procedure.

At step 406, an initial assessment workflow is provided, for example by the surgical system 200. The initial assessment workflow provides an initial assessment of the joint to be operated upon based on tracked poses of the bones of the joint. For example, the initial assessment workflow may include tracking relative positions of a tibia and a femur using data from the tracking system while providing real-time visualizations of the tibia and femur via a graphical user interface. The computing system 224 may provide instructions via the graphical user interface to move the tibia and femur to different relative positions (e.g., different degrees of flexion) and to exert different forces on the joint (e.g., a varus or valgus force). In some embodiments, the initial assessment workflow includes determine, by the surgical system 200 and based on data from the tracking system 222, whether the patient's joint has a varus or valgus deformity, and, in some embodiments, determining a magnitude of the deformity. In some embodiments, the initial assessment workflow may include collecting data relating to native ligament tension or native gaps between bones of the joint. In some embodiments, the initial assessment workflow may include displaying instructions to exert a force on the patient's leg to place the joint in a corrected state corresponding to a desired outcome for a joint arthroplasty procedure, and recording the relative poses of the bones and other relevant measurements while the joint is in the corrected state. The initial assessment workflow thereby results in collection of data that may be useful for the surgical system 200 or a surgeon in later steps of process 400.

At step 408, an implant planning workflow is provided, for example by the surgical system 200. The implant planning workflow is configured to facilitate users in planning implant placement relative to the patient's bones and/or planning bone cuts or other modifications for preparing bones to receive implant components. Step 408 may include generating, for example by the computing system 224, three-dimensional computer models of the bones of the joint (e.g., a tibia model and a femur model) based on the segmented medical images received at step 402. Step 408 may also include obtaining three-dimensional computer models of prosthetic components to be implanted at the joint (e.g., a tibial implant model and a femoral implant model). A graphical user interface can be generated showing multiple views of the three-dimensional bone models with the three-dimensional implant models shown in planned positions relative to the three-dimensional bone models. Providing the implant planning workflow can include enabling the user to adjust the position and orientation of the implant models relative to the bone models. Planned cuts for preparing the bones to allow the implants to be implanted at the planned positions can then be automatically based on the positioning of the implant models relative to the bone models.

The graphical user interface can include data and measurements from pre-operative patient data (e.g., from step 402) and from the initial assessment workflow (step 406) and/or related measurements that would result from the planned implant placement. The planned measurements (e.g., planned gaps, planned varus/valgus angles, etc.) can be calculated based in part on data collected via the tracking system 222 in other phases of process 400, for example from initial assessment in step 406 or trialing or tensioning workflows described below with reference to step 412.

The implant planning workflow may also include providing warnings (alerts, notifications) to users when an implant plan violates various criteria. In some cases, the criteria can be predefined, for example related to regulatory or system requirements that are constant for all surgeons and/or for all patients. In other embodiments, the criteria may be related to surgeon preferences, such that the criteria for triggering a warning can be different for different surgeons. In some cases, the computing system 224 can prevent the process 400 from moving out of the implant planning workflow when one or more of certain criteria are not met.

The implant planning workflow provided at step 408 thereby results in planned cuts for preparing a joint to receive prosthetic implant components. In some embodiments, the planned cuts include a planar tibial cut and multiple planar femoral cuts, for example as described above with reference to FIG. 1. The planned cuts can be defined relative to the virtual bone models used in the implant planning workflow at step 408. Based on registration processes from step 404 which define a relationship between tracked fiducial markers and the virtual bone models, the positions and orientations of the planned cuts can also be defined relative to the tracked fiducial markers, (e.g., in a coordinate system used by the tracking system 222). The surgical system 200 is thereby configured to associate the planned cuts output from step 408 with corresponding planes or other geometries in real space.

At step 410, a bone preparation workflow is provided, for example by the surgical system 200. The bone preparation workflow includes guiding execution of one or more cuts or other bone modifications based on the surgical plan created at step 408. For example, as explained in detail above with reference to FIGS. 2-3, the bone preparation workflow may include providing haptic feedback which constrains the surgical device 234 to a plane associated with a planned cut to facilitate use of the surgical device 234 to make that planned cut. In other embodiments, the bone preparation workflow can include automatically controlling the robotic device 220 to autonomously make one or more cuts or other bone modifications to carry out the surgical plan created at step 408. In other embodiments, the bone preparation workflow comprises causing the robotic device 220 to hold a cutting guide, drill guide, jig, etc. in a substantially fixed position that allows a separate surgical device to be used to execute the planned cut while being confined by the cutting guide, drill guide, jig, etc. The bone preparation workflow can thus include control of a robotic device in accordance with the surgical plan.

The bone preparation workflow at step 410 can also include displaying graphical user interface elements configured to guide a surgeon in completing one or more planned cuts. For example, the bone preparation workflow can include tracking the position of a surgical device relative to a plane or other geometry associated with a planned cut and relative to the bone to be cut. In this example, the bone preparation workflow can include displaying, in real-time, the relative positions of the surgical device, cut plane or other geometry, and bone model. In some embodiments, visual, audio, or haptic warnings can be provided to indicate completion or start of an event or step of the procedure, entry or exit from a state or virtual object, interruptions to performance of the planned cut, deviation from the planned cut, or violation of other criteria relating to the bone preparation workflow.

In some embodiments, step 410 is provided until all bone cuts planned at step 408 are complete and the bones are ready to be coupled to the implant components. In other embodiments, for example as shown in FIG. 4, a first iteration of step 410 can include performing only a portion of the planned cuts. For example, in a total knee arthroplasty procedure, a first iteration of step 410 can include making a tibial cut to provide a planar surface on the tibia without modifying the femur in the first iteration of step 410.

Following an iteration of the bone preparation workflow at step 410, the process 400 can proceed to step 412. At step 412 a mid-resection tensioning workflow or a trialing workflow is provided, for example by the surgical system 200. The mid-resection tensioning workflow is provided when less than all of the bone resection has been completed. The trialing workflow is provided when all resections have been made and/or bones are otherwise prepared to be temporarily coupled to trial implants. The mid-resection tensioning workflow and the trialing workflow at step 412 provide for collection of intraoperative data relating to relative positions of bones of the joint using the tracking system 222 including performing gap measurements or other tensioning procedures that can facilitate soft tissue balancing and/or adjustments to the surgical plan.

For example, step 412 may include displaying instructions to a user to move the joint through a range of motion, for example from flexion to extension, while the tracking system 222 tracks the bones. In some embodiments, gap distances between bones are determined from data collected by the tracking system 222 as a surgeon places the joint in both flexion and extension. In some embodiments, soft tissue tension or distraction forces are measured. Because one or more bone resections have been made before step 412 and soft tissue has been affected by the procedure, the mechanics of the joint may be different than during the initial assessment workflow of step 402 and relative to when the pre-operative imaging was performed. Accordingly, providing for intra-operative measurements in step 412 can provide information to a surgeon and to the surgical system 200 that was not available pre-operatively and which can be used to help fine tune the surgical plan.

From step 412, the process 400 returns to step 408 to provide the implant planning workflow again, now augmented with data collected during a mid-resection or trialing workflow at step 412. For example, planned gaps between implants can be calculated based on the intraoperative measurements collected at step 414, the planned position of a tibial implant relative to a tibia, and the planned position of a femoral implant relative to a femur. The planned gap values can then be displayed in an implant planning interface during step 408 to allow a surgeon to adjust the planned implant positions based on the calculated gap values. In various embodiments, a second iteration of step 408 to provide the implant planning workflow incorporates various data from step 412 in order to facilitate a surgeon in modifying and fine-tuning the surgical plan intraoperatively.

Steps 408, 410, and 412 can be performed multiple times to provide for intra-operative updates to the surgical plan based on intraoperative measurements collected between bone resections. For example, in some cases, a first iteration of steps 408, 410, and 412 includes planning a tibial cut in step 408, executing the planned tibial cut in step 410, and providing a mid-resection tensioning workflow in step 414. In this example, a second iteration of steps 408, 410, and 412 can include planning femoral cuts using data collected in the mid-resection tensioning workflow in step 408, executing the femoral cuts in step 410, and providing a trialing workflow in step 412. Providing the trialing workflow can include displaying instructions relating to placing trial implants on the prepared bone surfaces, and, in some embodiments, verifying that the trial implants are positioned in planned positions using the tracking system 222. Tracking data can be collected in a trialing workflow in step 412 relating to whether the trial implants are placed in acceptable positions or whether further adjustments to the surgical plan are needed by cycling back to step 408 and making further bone modifications in another iteration of step 410.

In some embodiments, executing process 400 can include providing users with options to jump between steps of the process 400 to enter a desired workflow. For example, a user can be allowed to switch between implant planning and bone preparation on demand. In other embodiments, executing process 400 can include ensuring that a particular sequence of steps of process 400 are followed. In various embodiments, any number of iterations of the various steps can be performed until a surgeon is satisfied that the bones have been properly prepared to receive implant components in clinically-appropriate positions.

As shown in FIG. 4, the process 400 includes step 414 where implantation of prosthetic components is facilitated. Once the bones have been prepared via step 410, the prosthetic components can be implanted. In some embodiments, step 414 is executed by the surgical system 200 by removing the robotic arm 232 from the surgical field and otherwise getting out of the way to allow a surgeon to fix the prosthetic components onto the bones without further assistance from the surgical system 200. In some embodiments, step 414 includes displaying instructions and/or navigational information that supports a surgeon in placing prosthetic components in the planned positions. In yet other embodiments, step 414 includes controlling the robotic arm 232 to place one or more prosthetic components in planned positions (e.g., holding a prosthetic component in the planned position while cement cures, while screws are inserted, constraining an impaction device to planned trajectory). Process 400 can thereby result in prosthetic components being affixed to modified bones according to an intra-operatively updated surgical plan.

Referring now to FIG. 5, a robotic device 500 is shown, according to an exemplary embodiment. In general, the robotic device 500 is configured to modify a patient's anatomy (e.g., femur, tibia, etc.). Robotic device 500 may be an exemplary embodiment of the robotic device 220 as shown in FIG. 2, and may be part of surgical system 200 as shown in FIG. 2. The robotic device 500 includes a base 502, a robotic arm 504, and a surgical device 506. The robotic device 500 may be communicably coupled to a tracking system and a computing system (e.g., tracking system 222 and computing system 224).

The base 502 provides a moveable foundation for robotic arm 504, allowing the robotic arm 504 and the surgical device 506 to be positioned and repositioned as needed relative to a patient. The base 502 may also contain power systems, computing elements, motors, and other electronic or mechanical systems necessary for the functions of the robotic arm 504 and the surgical device 506 described below.

As described above in reference to the robotic device 220 in FIG. 2, the robotic arm 504 is configured to support the surgical device 506 and provide a force as instructed by a computing system (e.g., computing system 224). In some embodiments, the robotic arm 504 allows a user to manipulate the surgical device 506 and provides force feedback to the user. In such an embodiment, the robotic arm 504 includes joints 508 and a mount 510 that includes motors, actuators, or other mechanisms configured to allow a user to freely translate and rotate the robotic arm 504 and surgical device 506 through allowable poses while providing feedback to constrain or prevent some movements of the robotic arm 504 and surgical device 506 as instructed by the computing system 224. In some embodiments, the robotic arm 504 is configured to move the surgical device 506 to a new pose automatically, without direct user manipulation, as instructed by computing system 224 in order to position the robotic arm 504 as desired and/or to complete certain surgical tasks, including modifications to a patient's anatomy (e.g., femur, tibia, etc.).

In some embodiments, the surgical device 506 is configured to cut, burr, grind, drill, partially resect, reshape, and/or otherwise modify a bone. The surgical device 506 may also include a holding arm or other support configured to hold an implant (e.g., acetabular cup, implant augment, etc.), or an impaction tool configured to provide impaction force to a cup implant. The surgical device 506 may also be, or include, any suitable cutting tool (e.g., a drill with a rotary bit, a drill with a spherical burr, a sagittal saw, a sagittal saw blade, a laser cutting device, etc.), and may be, or include, one of multiple tools interchangeably connected to the robotic device 500. For example, as shown in FIG. 5 the surgical device 506 may be a sagittal saw, comprising a housing 512, a handle 514, a sagittal saw blade 516, and a trigger mechanism 518. The housing 512 may be interchangeably connected to mount 510, and may be configured to support the handle 514, sagittal saw blade 516, and trigger mechanism 518. The housing 512 may also contain power systems, computing elements, motors, and other electronic or mechanical systems necessary for the functions of the surgical device 506. The handle 514 may extend from housing 512, and may be configured to allow the user to manipulate the surgical device 506. The handle 514 may be made of any material suitable for cleaning or sterilization. The sagittal saw blade 516 may be interchangeably connected to the housing 512, and may be aligned parallel with the housing 512, or perpendicular to the housing 512 axis. Trigger mechanism 518 may be connected to the housing 512, and can be configured to be pressed (depressed), released, held in place, double-pressed (e.g., pressed, released, and then pressed again in quick succession (e.g., within one second)), or any combination thereof. The trigger mechanism 518 may also be made of any material suitable for cleaning or sterilization, and may interact with the electronic or mechanical systems necessary for the functions of the surgical device 506 located in the housing 512.

Robot Control with Force Feedback

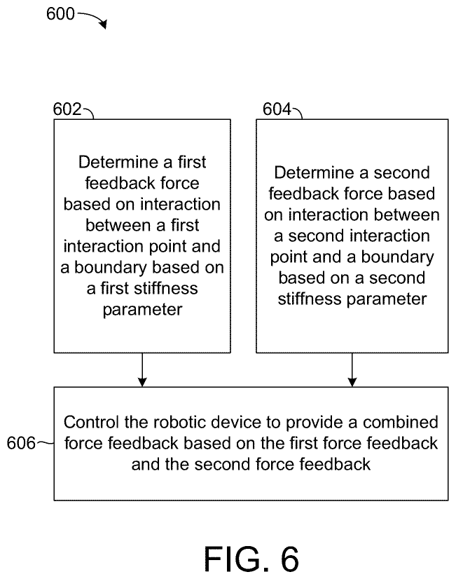

Referring now to FIG. 6, a flowchart of a process 600 is shown, according to some embodiments. Process 600 can be executed by the computing system 224, for example as part of step 310 of process 300 and/or as part of step 410 of process 400. Process 600 can be implemented using surgical system 200 and/or robotic device 220 or robotic device 500 as described above, or other implementations of robotically-assisted surgical systems, in various embodiments.

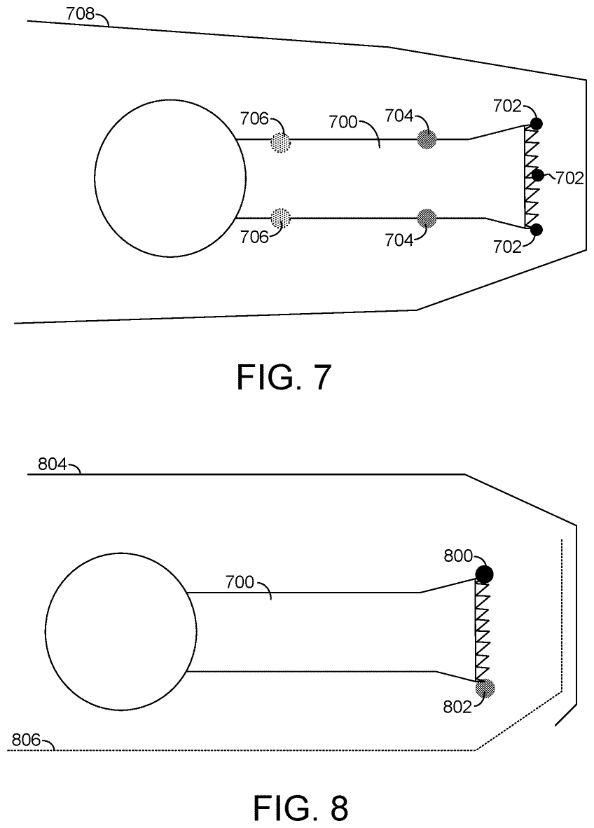

In the embodiment of FIG. 6, multiple interaction points (e.g., HIPs as discussed above) can be defined relative to a surgical tool or other end effector of a robotic device. For example, a first interaction point may be at the tool center point (TCP), for example at a cutting tip of a burr, cutting edge of a saw, etc. (e.g., surgical device 234, surgical device 506). A second interaction point may be spaced apart from the first interaction point, for example located along a shaft of the cutting tool or elsewhere on the cutting tool, end effector, robotic arm, or the like. Examples of such embodiments are shown in FIGS. 7, 8, and 10, described in detail below with reference thereto. Process 600 can include determining such interaction points and tracking such interaction points in space relative to target patient anatomy, for example relative to a bone to be modified (e.g., resected) using the surgical tool as a cutting tool or cutting guide. Such tracking can be performed using optical tracking or other tracking modality as described above.

At step 602, a first feedback force is determined based on an interaction between a first interaction point and a boundary based on a first stiffness parameter. Step 602 can include tracking a position of the first interaction point relative to a boundary (haptic boundary, control boundary, virtual geometry, etc.), for example based on tracked positions of the surgical tool and a target anatomical structure associated with the boundary. The first feedback force can be determined such that the first feedback force is configured to constrain the first interaction point from crossing the boundary, with a magnitude of the first feedback force determined based on the first stiffness parameter. For example, the first feedback force can be provided in a direction pointing away from (e.g., normal to) the boundary and with a magnitude based on a spring-force formula using the first stiffness parameter as a scalar weight, such as

$${F}_{1} = {k}_{1} ( \frac{1}{{x}_{1}} )$$

where k₁ is the first stiffness parameter, x₁ is a distance between the first interaction point and the boundary, and F₁ is the first feedback force (e.g., limited to some maximum value as x₁ approaches zero). As another example, the first stiffness parameter can define an exponential relationship, for example according to a function of the form F₁=(1/x₁)^{k}^{₁} (e.g., limited to some maximum value as x₁ approaches zero). The present disclosure contemplates may such examples by which step 602 can determine a first feedback force based on an interaction between a first interaction point and a boundary based on a first stiffness parameter. In other embodiments, the first stiffness parameter is provided as a look-up table storing force values, stiffness values, etc. for different distance values, which can be retrieved based on the distance between the first interaction point and the boundary to obtain a feedback force associated with providing a first stiffness profile. Various details relating to determining a distance and direction between an interaction point and a boundary (e.g., represented as a surface mesh) are provided in U.S. Pat. No. 8,010,180, the entire disclosure of which is incorporated by reference herein.

At step 604, a second feedback force is determined based on an interaction between a second interaction point and a boundary based on a second stiffness parameter. Step 604 can include tracking a position of the second interaction point relative to a boundary (haptic boundary, control boundary, virtual geometry, etc.), for example based on tracked positions of the surgical tool and a target anatomical structure associated with the boundary. The second feedback force can be determined such that the second feedback force is configured to constrain the second interaction point from crossing the boundary, with a magnitude of the second feedback force determined based on the second stiffness parameter. For example, the second feedback force can be provided in a direction pointing away from (e.g., normal to) the boundary and with a magnitude based on a spring-force formula using the first stiffness parameter as a scalar weight, such as

$${F}_{2} = {k}_{2} ( \frac{1}{{x}_{2}} )$$

where kₛ is the second stiffness parameter, x₂ is a distance between the first interaction point and the boundary, and F₂ is the first feedback force (e.g., limited to some maximum value as x₂ approaches zero). As another example, the first stiffness parameter can define an exponential relationship, for example according to a function of the form F₂=(1/x₂)^{k}^{₂} (e.g., limited to some maximum value as x₂ approaches zero). In other embodiments, the second stiffness parameter is provided as a look-up table storing force values, stiffness values, etc. for different distance values, which can be retrieved based on the distance between the second interaction point and the boundary to obtain a feedback force associated with providing a second stiffness profile. The present disclosure contemplates may such examples by which step 604 can determine a second feedback force based on an interaction between a second interaction point and a boundary based on a second stiffness parameter.

In some embodiments, the second stiffness parameter is different than (e.g., greater than, less than) the first stiffness parameter. Lower values of the stiffness parameter may be associated with a less stiff (more pliable) interaction with the boundary, for example where a user experiences an ability to depress, push slightly into, or otherwise feel the boundary as relative soft (to a degree defined by the value of the stiffness parameter). Higher values of the stiffness parameter may be associated with a stiffer interaction with the boundary, i.e., less or no experience of being able to depress or push into the boundary, abrupt feedback, or otherwise feeling the boundary as hard, rigid, etc. (again, to a degree defined by the value of the stiffness parameter).

At step 606, a robotic device is controlled to provide a combined force feedback based on the first force feedback and the second force feedback. Step 606 can include adding the first force feedback and the second force feedback (e.g., as vectors) and determining a combined force feedback to be applied (e.g., based on both the magnitudes and directions of the first force feedback and the second force feedback). Step 606 can then include determining control signals for motorized joints or other actuators of the robotic device to generate the determined combined force feedback, and providing such control signals to the motorized joints or other actuators so as to cause the motorized joints or other actuators to provide the combined force feedback at the end effector of the robotic device (e.g., a surgical tool held by the robotic device).

By generating a combined force based on the first force feedback and the second force feedback, which are in turn based on the first stiffness parameter and the second stiffness parameter, process 600 can provide a user experiencing such force feedback with a different perception of the stiffness of the boundary depending on whether the first interaction point or the second interaction is interacting with the boundary. For example, in some embodiments, the first stiffness parameter associated with the first interaction point (e.g., at a tool tip) is greater than the second stiffness parameter associated with the second interaction point (e.g., at a tool shaft), such that, as the first interaction point moves toward, arrives at, or otherwise interacts with the boundary, the first feedback force is determined in an manner which is stiffer (more rigid, harder, greater force) as compared to the second feedback force as the second interaction point moves toward, arrives at, or otherwise interacts with the boundary. Various other differences in force feedback as experienced by the user can be achieved by using different stiffness profiles or functions, for example different look-up tables or plots, etc. of force or stiffness values to be provided for different interaction points at different distances to different boundaries, leading to a high degree of variability of force feedback provided for different points, different boundaries, etc. according to the various examples herein and adaptations thereof.

The robotic device can be controlled in step 606 such that the combined force feedback causes the user to experience a stiffer boundary with the first interaction point (e.g., the tool tip) and a relatively softer boundary with the second interaction point (e.g., the tool shaft), including, in some scenarios, when both interaction points are simultaneously interacting with the boundary. Such an embodiment may be desirable in order to provide a user with confidence that the cutting-end of a surgical tool is rigidly constrained to a virtual object (e.g., associated with a planned bone resection as described above) while also experiencing a softness with respect to another point on the surgical tool interacting with a boundary (e.g., reflecting that the shaft of the surgical tool may be near soft tissue, facilitating tool alignment, enabling a user to feel the difference between first force feedback associated with the first interaction point and second force feedback associated with the second point, etc.). For example, such an embodiment may be desirable to provide a rigid (high stiffness) constraint that constrains the cutting-end of a surgical tool from contacting soft tissue, retractors, trackers (e.g., fiducial trees 240, 241), etc., while softer (less stiff) constraints associated with one or more points on a shaft of the surgical tool (or elsewhere on device 506 in the example of FIG. 5) can permit some degree of contact between the shaft and soft tissues, retractors, or the like.

Various such advantages can be obtained by selection of different values of the first stiffness parameter and the second stiffness parameter and execution of process 600. Some embodiments additional including additional interaction points associated with the same or additional stiffness parameters. While the examples herein refer to stiffness parameters, other parameters or functions for determining force feedback can vary across different interaction points, across various boundaries, in response to various events and adjustments, and/or in response to other logic described in the embodiments herein as being for stiffness adjustment. All such variations are within the scope of the present disclosure.

Referring now to FIG. 7, a diagram of an end effector of a robotic device having associated interaction points relative to a virtual boundary is shown, according to some embodiments. FIG. 7 shows an arrangement which can enable some embodiments of process 600, for example. As shown in FIG. 7, an end effector 700 (e.g., surgical device 234, surgical device 506, bone saw) is illustrated along with three first interaction points 702 at a cutting end of the end effector 700, a pair of second interaction points 704 at an end effector 700 at a first distance from the cutting end of the end effector 700, and a pair of third interaction points 706 at a third distance from the cutting end of the end effector 700. FIG. 7 also illustrates a virtual boundary 708, for example a haptic boundary, control boundary, etc. associated with a planned bone resection to be made using the end effector 700. The end effector 700 is coupled to a robotic device, for example as in robotic device 500 and/or as for surgical device 234 described above.

Force feedback can be provided to the end effector 700 (e.g., to a user manipulating the end effector) according to an implementation of process 600. First force feedback can be determined based on a first stiffness parameter when one or more of the first interaction points 702 interact with (e.g., come near, touch, start to cross, etc.) the virtual boundary 708, second force feedback can be determined based on a second stiffness parameter (e.g., different than the first stiffness parameter) when one or both of the second interaction points 704 interact with the virtual boundary, and third force feedback can be determined based on a third stiffness parameter (e.g., different than the first and second stiffness parameters) when the third interaction points 706 interact with the virtual boundary. One of more of the first force feedback, the second force feedback, and/or the third force feedback can be zero or non-zero at a given time based on the positions of the first interaction points 702, the second interaction points 704, and the third interaction points 706 relative to the boundary. Combined force feedback can then be generated and provided to the end effector 700 (via a robotic device coupled to the end effector) according to teachings of step 606 described above.

In some embodiments as shown in FIG. 7, the stiffness parameter(s) associated with the second interaction points 704 and the third interaction points 706 are lower than the stiffness parameter associated with the first interaction points 702. In such embodiments, combined force feedback is generated such that the boundary feels relatively less stiff (softer) when the non-cutting portions of the end effector 700 (e.g., shaft of the end effector 700 where the second interaction points 704 and the third interaction points 706 are positioned) interact with the boundary 708 as compared to the boundary feeling relatively stiff (harder) when the cutting end of the end effector 700 interacts with the boundary.

In other embodiments, the stiffness parameter(s) associated with the second interaction points 704 and the third interaction points 706 are higher than the stiffness parameter associated with the first interaction points 702. In such embodiments, combined force feedback is generated such that the boundary feels relatively stiffer (harder) when the non-cutting portions of the end effector 700 (e.g., shaft of the end effector 700 where the second interaction points 704 and the third interaction points 706 are positioned) interact with the boundary 708 as compared to the boundary feeling relatively less stiff (softer) when the cutting end of the end effector 700 interacts with the boundary.

While FIG. 7 shows a particular arrangement of interaction points along a saw, it should be understood that the teachings here can be adapted to different arrangement interaction points and different surgical tools, for example burrs, drills, reamers, and the like.

Referring now to FIG. 8, another illustration of the end effector 700 is shown. The end effector 700 is coupled to a robotic device, for example as in robotic device 500 and/or as for surgical device 234 described above. In the embodiment of FIG. 8, a first interaction point 800 and a second interaction point 802 are defined relative to the end effector 700, for example positioned at different positions on a cutting end of the end effector 700 as illustrated in FIG. 8. FIG. 7 also illustrates a first virtual boundary 804 and a second virtual boundary 806, for example corresponding defined based on a surgical plan according to the teachings above. The first virtual boundary 804 may be a different shape than the second virtual boundary 806, may be moveable relative to the second virtual boundary 806 (e.g., based on tracked movement of bone, soft tissue, retractors, etc.), and/or may otherwise be distinct from the second virtual boundary 806. In the example shown, force feedback can be generated based on a first stiffness parameter when the first interaction point 800 interacts with the first virtual boundary 804 and based on a second stiffness parameter when the second interaction point 802 interacts with the second virtual boundary 806. In some embodiments, force feedback is also generated based on a third stiffness parameter when the first interaction point 800 interacts with the second virtual boundary 806 and a fourth stiffness parameter when the second interaction point 802 interacts with the first virtual boundary 804. FIG. 8 thus illustrates that the teachings of at least FIG. 6 herein can be adapted for use with multiple virtual boundaries, where different stiffness parameters are associated with different combinations of interaction points and virtual boundaries so as to tune the stiffness of force feedback based on virtual boundaries as may be desirable for different surgical procedures, based on different surgeon preferences, different surgical tools, based on different user selections and inputs requesting activation and deactivation of different boundaries, etc.

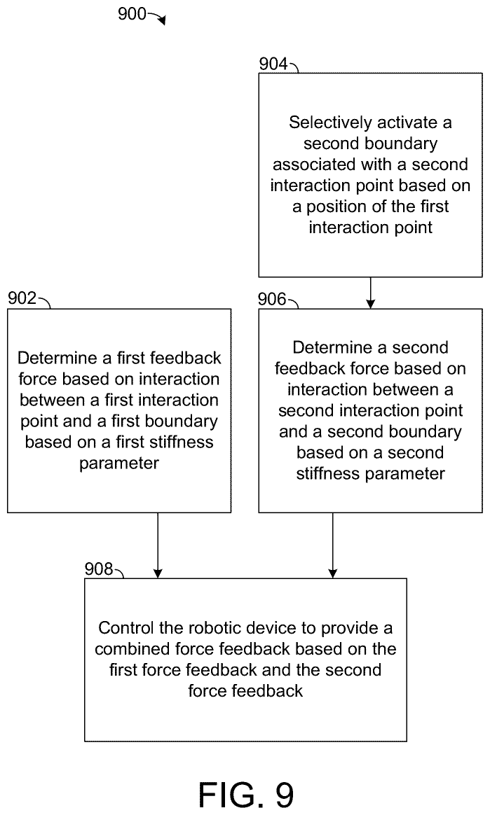

Referring now to FIG. 9, a flowchart of a process 900 relating to providing force feedback with a robotic device is shown, according to some embodiments. Process 900 can be executed by the computing system 224, for example as part of step 310 of process 300 and/or as part of step 410 of process 400. Process 900 can be implemented using surgical system 200 and/or robotic device 220 (e.g., robotic device 500) as described above, or other implementations of robotically-assisted surgical systems, in various embodiments.

At step 902, a first force feedback is determined based on an interaction between a first interaction point and a first boundary, and, in some embodiments, based on a first stiffness parameter. Step 902 can be implemented according to the description of step 602 above.

At step 904, a second boundary associated with a second interaction point is selectively activated based on a position of the first interaction point. Activating the second boundary in step 904 can be performed responsive to the position of the first interaction point satisfying one or more criteria. For example, step 904 can include indicating a criterion as satisfied when the first interaction point is within a threshold distance of an anatomical target (e.g., a patient's bone, a particular point or surface of the patient's bone, etc.). As another example, step 904 can include indicating a criterion as satisfied when the first interaction point has reached the anatomical target (e.g., when the surgical tool starts to interact with the anatomical target). As another example, step 904 can include indicating a criterion as satisfied when the first interaction point is within an activation zone spaced apart from the anatomical target, for example such that the criterion is unsatisfied if the first interaction point is closer to the anatomical target than the activation zone. As yet another example, step 904 can include indicating a criterion as satisfied based on a change in the position of the first interaction point, for example if a velocity of the first interaction point exceeds a threshold (or reduces to lower than a threshold), if a direction of the change in position is within a range of directions (e.g., is substantially toward a target, deviates from a planned trajectory, etc.), if the change in position follows a defined pattern (trajectory, shape, etc.), etc. Various such criterion can be used in step 904 based on a position of the first interaction point in order to determine when to activate the second boundary.

Activation of the second boundary in step 904 can include transitioning from a state in which force feedback is provided via robotic device based on interactions with the first boundary to a state in which force feedback is provided via the robotic device based on interactions with the first boundary and the second boundary. For example, before selective activation of the second boundary in step 904, one or more interaction points can cross the second boundary without force feedback being provided (as the second boundary is in an inactive state). Activation of the second boundary in step 904 transitions the second boundary into an active state where the second boundary can be used to determine force feedback as described below with reference to steps 906 and 908. In some embodiments, process 900 includes deactivating the second boundary at such time as one or more criteria assessed in step 904 become unsatisfied.

At step 906, a second feedback force is determined based on interaction between a second interaction point and a second boundary, in some embodiments based on a second stiffness parameter. The second feedback force can be determined as described above for step 604, adapted to the embodiments of FIG. 9 in which the second feedback force is determined based on a second boundary activated via step 904 (e.g., rather than the first boundary used in step 902).

At step 908, a robotic device is controlled to provide a combined force feedback based on the first force feedback and the second force feedback. Step 908 can be provided as described above for step 606, adapted here such that the combined force feedback includes a combination of the first force and the second force if the second boundary has been activated in step 904 (and can be the first force feedback when the second boundary has not been activated in step 904).

Process 900 can thereby provide force feedback associated with a first interaction point and a first boundary, and selectively further based on a second interaction point and a second boundary if activated (e.g., activated responsive to position of the first interaction point satisfying a criterion). As described above, the force feedback associated with the first interaction point and can be provided with a different stiffness than the force feedback associated with the second interaction point.

In some implementations, process 900 enables a first interaction point at a cutting tip (e.g., tool center point) of a cutting tool to be constrained to a first boundary based on a first stiffness parameter, while a second interaction point along the shaft of the cutting tool is free to move throughout space (e.g., rotate about the cutting tip/first interaction point) until such time as the first interaction point is positioned at a threshold position (e.g., within a threshold distance of an anatomical target, within a threshold distance of virtual target, within an activation region defined relative to the first boundary, etc.); the unconstrained range of motion of the second interaction point can facilitate a surgeon in moving the first interaction point to the threshold position (e.g., allowing the shaft of the tool to be rotated to avoid retractors, soft tissue, etc. in the surgical field). Upon the first interaction point reaching the threshold position (or satisfying other criteria according to various embodiments), the second boundary is activated such that different points on the cutting tool become constrained by different boundaries, for example further based on different stiffness parameters. The second boundary can be defined based on objects or patient tissue which may be desirable to avoid contacting with the cutting tool and/or based on positions which ensure desirable orientation of the cutting tool (e.g., to improve the quality of bone cutting, etc.), with the first boundary can be defined based on a desired bone resection or other modification of an anatomical target. Combined force feedback according to process 900 can advantageously provide a workflow for surgical approach and bone cutting (or other surgical intervention) in an user friendly and intuitive manner.

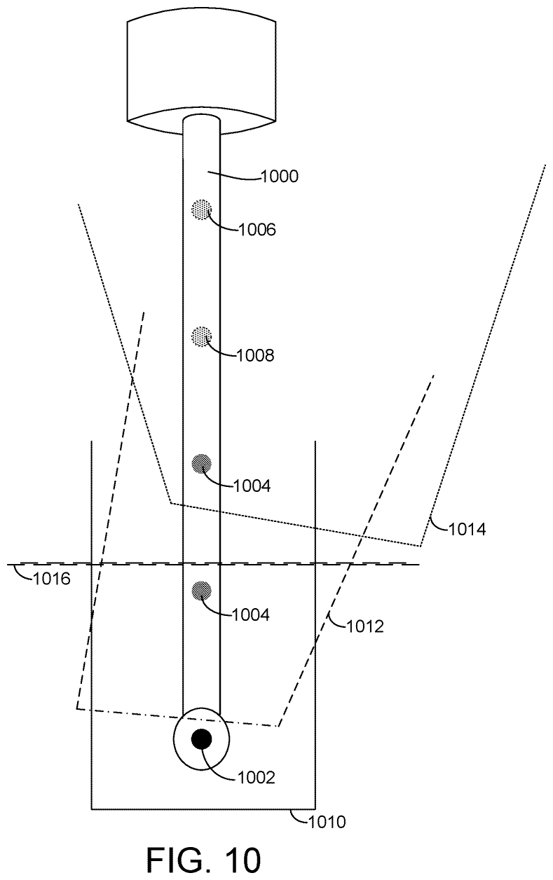

Referring now to FIG. 10, a diagram of a surgical tool 1000 used with a robotic device (e.g., robotic device 500) and multiple virtual boundaries is shown, according to some embodiments. FIG. 10 illustrates an example implementation of process 900, in some embodiments. FIG. 10 shows a surgical tool 1000 (e.g., surgical device 234, surgical device 506, burr) and interaction points defined relative to the surgical tool 1000, in particular a first interaction point 1002 (e.g., tool center point) at a cutting tip of the surgical tool 1000, two second interaction points 1004 at points along a shaft of the surgical tool 1000, and two third interaction points 1006 at points along the shaft of the surgical tool 1000. The two second interaction points 1004 are shown as being between the third interaction points 1006 and the first interaction point 1002. Various interaction points can be defined along, near, around, etc. the surgical tool 1000 (or other elements of a robotic device or system) according to various embodiments (e.g., on housing 512, handle 514, mount 510, on one or more segments of the arm 504, etc. with reference to FIG. 5). The surgical tool 1000 is coupled to a robotic device, for example as for surgical devices 234 and 506 described above.

FIG. 10 further shows a first virtual boundary 1010, a second virtual boundary 1012, and a third virtual boundary 1014. In the example of FIG. 10, force feedback can be provided to the surgical tool 1000 in response to first interactions between the first interaction point 1002 and the first virtual boundary 1010, second interactions between the second interaction points 1004 and the second virtual boundary 1012, and third interactions between third interaction points and the third virtual boundary 1014.

FIG. 10 further shows an activation line 1016. The first interaction point 1002 may cross the activation line 1016 to reach the position shown in FIG. 10 from a starting position outside the surgical tool (e.g., above the activation line 1016 from the perspective of FIG. 10). In some embodiments, for example as an implementation of step 904 of FIG. 9, the second virtual boundary 1012 and the third virtual boundary 1014 can be inactive (e.g., not used in generating force feedback) before the first interaction point 1002 crosses the activation line 1016 and can be activated to provide force feedback in response to the first interaction point crossing the activation line 1016. The second boundary 1012 and/or the third boundary 1014 can thereby be activated based on a position of the first interaction point 1002 as in process 900 described above.

Advantageously, each section of the tool can thereby have its own location-based haptics that activated based on tool position, according to various embodiments. Different boundaries, stiffness parameters, other feedback parameters, etc. can be provided for each section of the tool (e.g., of surgical device 234, tool 1000). For example, the first boundary 1010 may provide constraint on cutting by the tool 1000 within a bone (e.g., defining a planned resection), the second boundary 1012 can constrain a portion of the shaft of the tool 1000 (e.g., as represented by second interaction points 1004) which extends into a cavity in the bone (e.g., a cavity created by operation of the tool 1000), while the third boundary 1014 can constrain a portion of the shaft of the tool 1000 which remains outside of the bone (e.g., external to an original bone surface, not inserted into a cavity in the bone).

The multiple boundaries can have a variety of shapes in various embodiments, for example in one, two, or three dimensions, for example funnel shapes, cone shapes, cylindrical shapes, prisms, planes, lines, etc. in various embodiments, with the first boundary 1010, the second boundary 1012, and the third boundary 1014 have similar shapes or different shapes in various embodiments. In some embodiments, the first boundary 1010 has a rectangular shape (e.g., a rectangular prism volume, a planar surface) while the second boundary 112 is cylindrical. All variations on such combinations are within the scope of the present disclosure.

In some embodiments, the processes herein include determining the location for boundaries, for example the second boundary 1012 and/or the third boundary 1014 to avoid structures (objects, devices, anatomical features, etc.) in the surgical environment. For example, the second boundary 1012 and/or the third boundary 1014 may be defined based on a location of one or more retractors, for example a pre-programmed or user-selected location and/or a location determined from machine learning, computer vision, selection with a tracked probe, etc. in various embodiments. The second boundary 1012 and/or the third boundary 1014 may additionally or alternative be defined based on location of soft tissue, for example one or more ligaments. In combination with other teachings herein, the stiffness of interactions with such boundaries may different for different points on a surgical tool, for example such that a shaft of the tool has relatively soft, pliable, etc. haptic interactions at or near soft tissue and/or retractors as compared to a cutting end of the surgical tool (which may have hard, rigid, etc. haptic interactions to prevent contact between the cutting end and such structures). Constraints on different sections of a surgical tool can thereby be robotically implemented to promote desirable behavior for particular sections of the surgical tool.

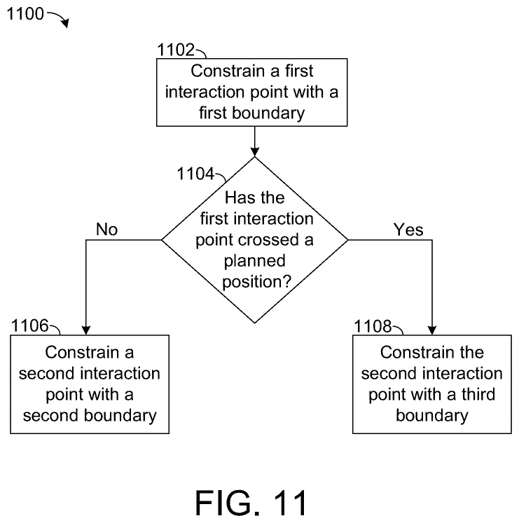

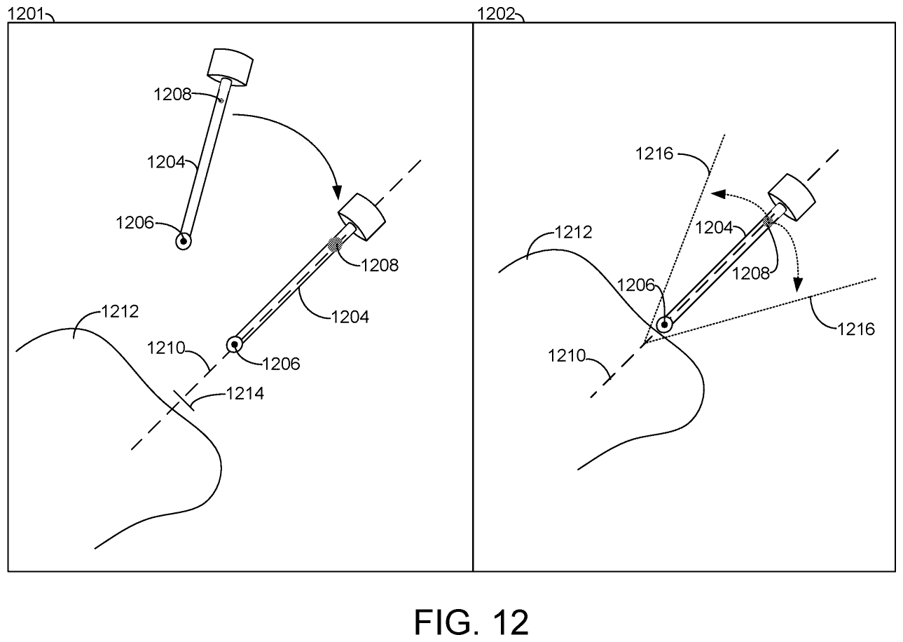

Referring now to FIG. 11, a flowchart of a process 1100 for controlling a robotic device is shown, according to some embodiments. Process 1100 can be executed by the computing system 224, for example as part of step 310 of process 300 and/or as part of step 410 of process 400. Process 1100 can be implemented using surgical system 200 and/or robotic device 220 as described above, or other implementations of robotically-assisted surgical systems, in various embodiments.

At step 1102, a first interaction point is constrained to a first boundary. The first interaction point can correspond to a tool center point of a surgical device 234, for example. Constraining the first interaction point to the first boundary can include controlling a robotic device to provide force feedback that resists movement of the first interaction point from crossing or otherwise deviating from the first boundary, for example according to various teachings described elsewhere herein.

At step 1104, a determination is made as to whether the first interaction point crossed a planned position. The planned position can be specified by a surgical plan, for example based on a planned bone resection to be made by the surgical device during a surgical operation. For example, the planned position can be a position spaced apart from a bone to be resected by a threshold amount, a position on a pre-surgical surface of the bone, a position at a particular depth into the bone along the planned resection, or other planned position in various embodiments. Step 1104 can include determine a position of the first interaction point relative to the planned position based on tracking of the surgical tool and a patient bone or other anatomy, for example using optical tracking, joint data of a robotic device, etc.

If the first interaction point has not crossed the planned position (e.g., since a beginning of a corresponding stage of the surgical procedure) (“No” from step 1104), the second interaction point associated with the surgical tool is constrained with a second boundary in step 1106. The second interaction point can be spaced apart from the first interaction point, for example positioned along a shaft of the surgical device (e.g., bur, saw, cutting tool). Step 1106 can include executing process 600 or process 900, for example. Force feedback is provided in step 1106 based on an interaction between the second interaction point and a second boundary. In some embodiments, step 1106 includes constraining the second interaction point with the second boundary while continuing to constrain the first interaction point with the first boundary (as from step 1102).

If the first interaction point has crossed the planned position (“Yes” from step 1104), the second interaction point associated with the surgical tool is constrained with a third boundary in step 1108. The third boundary is different than the second boundary (e.g., in shape, location, size, stiffness, or other parameter). The first boundary may be different from both the third boundary and the second boundary or may be the same as one of the second boundary or the third boundary, in various embodiments. Force feedback is provided in step 1108 based on interaction between the second interaction point and the third boundary. In some embodiments, step 1108 includes constraining the second interaction point with the third boundary while continuing to constrain the first interaction point with the first boundary, for example according to teachings of process 600 or process 900. In other embodiments, step 1108 includes constraining the second interaction point with the third boundary while abstaining from constraining the first interaction point with the first boundary (e.g., deactivating the first boundary in response to the first interaction point crossing the planned position).

Accordingly, process 900 provides for the second interaction point to be constrained by a second boundary before the first interaction point crosses a planned position and constrained with a third boundary after the second interaction point crosses the third boundary. Process 900 can thus include switching, for generation of feedback based on the second interaction point, from the second boundary to the third boundary in response to the first interaction point crossing the planned position. As one example implementation, the resulting force feedback may be advantageous in a scenario in which the shaft of a surgical tool should be constrained to a particular alignment for a first portion of a resection and a different alignment or degree of freedom for a second portion of a resection, based on a complex geometry of a planned resection (e.g., cutting a curved shape, segment around a corner, etc.), with a change in the boundary constraining the shaft occurring part way through the resection according to an example implementation of process 1100. Various other examples are also within the scope of process 1100, in various embodiments.

Referring now to FIG. 12, a storyboard-style illustration of a surgical tool constrained to virtual boundaries is shown, according to some embodiments. FIG. 12 illustrates an example implementation of process 1100 of FIG. 11, showing a first frame 1201 corresponding to at least step 1106 and a second frame 1202 corresponds to at least step 1108, as described further in the following passages.

The first frame 1201 illustrates alignment of a surgical tool 1204 (e.g., surgical device 234) to a virtual boundary 1210. The virtual boundary 1210 is shown as a line (e.g., one dimensional boundary), where the surgical tool 1204 is moved into alignment with the line, i.e., such that a first interaction point 1206 at the cutting tip of the surgical tool 1204 is positioned on the virtual boundary 1210 and a second interaction point along a shaft of the surgical tool 1204 is also positioned on the virtual boundary 1210. Alignment to the virtual boundary 1210 as in the first frame 1201 can be performed automatically by a robotic device holding the surgical tool 1204 (e.g., a robotic system as described in detail above), and/or via various other guidance provided to a user of the surgical device.

Upon alignment to the virtual boundary 1210 as in the first frame 1201, both the first interaction point 1206 and the second interaction point 1208 can be constrained to the virtual boundary 1210. In the example shown where the virtual boundary 1210 is a line, the surgical tool 1204 can be manipulated to move along the line, while a robotic device provides force feedback constraining both the first interaction point 1206 and the second interaction point 1208 from deviating from the line (i.e., the virtual boundary 1210). The virtual boundary 1210 is shown as being defined relative to a bone 1212 such that the virtual boundary 1210 can facilitate modification of the bone 1212 using the surgical tool 1204.

A planned position 1214 (e.g., threshold position, activation threshold, etc.) along the virtual boundary 1210 is also illustrated in the first frame 1201. In the first frame, the first interaction point 1206 has not yet reached or crossed the planned position 1214 (i.e., “No” at step 1104 in process 1100); as such, the second interaction point 1208 is constrained based on an interaction between the second interaction point 1208 and the virtual boundary 1210 (e.g., as in step 1106).

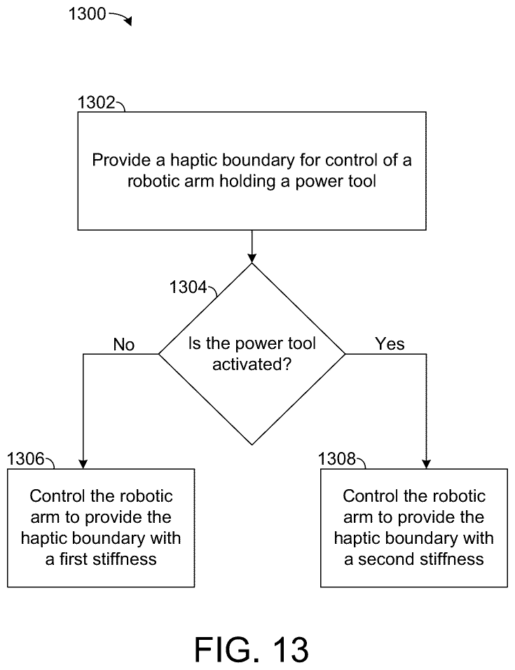

To transition from the first frame 1201 to the second frame 1202, the surgical tool 1204 is translated along the virtual boundary 1210 until the planned position 1214 reaches (crosses) the planned position 1214. Upon reaching (crossing) the planned position 1214 (i.e., in response to the result of step 1104 becoming “Yes,”), a transition is provided from the first frame 1201 in which the second interaction point 1208 is constrained by the virtual boundary 1210 to the second frame 1202 in which the second interaction point 1208 is instead by an additional virtual boundary 1216. In other embodiments, the transition from the first frame 1201 to the second frame 1202 is provided in response to activation of power to the surgical tool 1204 (e.g., as adapted from teachings of FIG. 13 described below).

As illustrated in the second frame, the additional virtual boundary 1216 is a cone or funnel shape providing the second interaction point with a different available range of motion as a compared to the virtual boundary 1210. In the example shown, the additional virtual boundary 1216 provides an additional degree of freedom for the second interaction point, in particular such that the second interaction point is allowed to rotate around the first interaction point 1206 so long as the second interaction point stays within the additional virtual boundary 1216. Other shapes, geometries, dimensionalities, etc. for the virtual boundary 1210 and the additional virtual boundary 1216 can be implemented in various embodiments.

Accordingly, as illustrated, the shaft of the surgical tool is maintained in alignment with a target axis for a bone modification during the first frame 1201, until the cutting end of the tool arrives at the bone, for example to facilitate a user in moving the tool into an appropriate starting position for a planned bone modification. Upon the tip of the tool reaching the planned position (i.e., a position set in a surgical plan), the shaft of the tool is given a degree of freedom to move within the additional boundary 1216, thereby facilitating a user in completing the planned bone modification while still providing force feedback for guidance of the planned bone modification. Different variations of the boundary shapes illustrated in FIG. 12 can facilitate different types of bone modifications as useful in different surgical procedures. Accordingly, in the example show, switching to the additional boundary provides the shaft of the tool with a larger range of motion as compared to before the switch; in other embodiments, switching to the additional boundary reduces the range of motion and/or removes a degree of freedom as compared to before the switch.

Referring now to FIG. 13, a flowchart of a process 1300 for controlling a robotic system is shown, according to some embodiments. Process 1300 can be executed by the computing system 224, for example as part of step 310 of process 300 and/or as part of step 410 of process 400. Process 1300 can be implemented using surgical system 200 and/or robotic device 220 as described above, or other implementations of robotically-assisted surgical systems, in various embodiments. Process 1300 can be provided in combination with process 600, process 900, or any of the various teachings provided herein.

At step 1302, a haptic boundary (virtual boundary, haptic object, control object, virtual geometry, etc.) is provided for controlling a robotic arm that holds a power tool or guide (e.g., drill guide, cutting guide, jig, etc.) for use with a power tool provided separate from the robotic arm (e.g., as a handheld tool for use with the guide held by the robotic arm). The haptic boundary can be provided according to a surgical plan and used to constrain movement of the power tool or guide, for example as described with reference to FIGS. 3-4. In some embodiments, step 1302 is provided by the robotic arm 232 and the surgical device 234 shown in FIG. 2. The power tool (e.g., surgical device 234) can be a powered saw, for example a reciprocating saw which powers oscillation of a serrated blade of the saw when power to the saw is activated. The power tool can be a burr, for example having a cutting tip which is rotated by power to the burr. The power tool can be a drill, for example having a drill bit which is rotated by power to the drill. The power tool can be an electrocautery device, laser cutter, ultrasonic cutter, and/or other type of cutting tool which coverts electrical power to such tool to mechanical, electrical, sonic or other form of energy which can cut, resect, ablate, or otherwise modify patient tissue.

At step 1304, a determination is made as to whether the power tool is activated. The power tool is active when a powered cutting component thereof is operating (e.g., a saw is reciprocating, a burr is rotating, a drill bit is rotating, a laser cutter is emitting a laser) as contrasted to an inactive state (the saw blade, burr tip, drill bit is static relative to a body of the tool; a laser cutter is not emitting a laser, etc.). In some embodiments, the power tool (e.g., surgical device 234 with reference to FIG. 2) can include a trigger, button, or other input located on or near the power tool (e.g., on a robotic arm) which can be engaged by a user to activate power to the power tool. In some embodiments, a switch, button, foot pedal, etc. is provided elsewhere in the surgical environment and/or via a graphical user interface of a surgical robotics system, and can be engaged by a user to activate or deactivate power to the power tool. The determination can be made based on electronic signals associated with engagement of the user of the trigger, button, or other input to activate the power tool as may be present in a given embodiment.

If the power tool is not activated (“No” at step 1304), the process 1300 proceeds to step 1306 where the robotic arm is controlled to provide the haptic boundary with a first stiffness. For example, step 1306 can include generating haptic feedback (force feedback, etc.) as a function of a position of the robotic arm (e.g., of the power tool or guide) (e.g., a haptic interaction point defined on the power tool or on the guide) relative to the haptic boundary and further based on a first stiffness parameter. The first stiffness parameter can be determinative of how stiff (hard, rigid, solid, etc.) the haptic boundary feels to a user of the power tool and the robotic arm (e.g., via interaction between the power tool and a guide held by the robotic arm in relevant embodiments).

Various formulations are possible for determining an amount of force to provide, by the robotic arm, to constrain the power tool with the haptic boundary with a first stiffness. In some embodiments, a feedback force can be provided in a direction pointing away from (e.g., normal to) the boundary and with a magnitude based on a spring-force formula using a first stiffness parameter as a scalar weight, such as having the form F=k₁ (1/x) where k₁ is the first stiffness parameter, x is a distance between an interaction point and the boundary, and F is a feedback force. As another example, the first stiffness parameter can define an exponential relationship, for example according to a function of the form F=(1/x)^{k}^{₁}. Various such formulations in which the stiffness of the haptic boundary can be adjusted by changing the value of a stiffness parameter can be used in various implementations of process 1300.

If the power tool is activated (“Yes” at step 1306), the process 1300 proceeds to step 1306 where the robotic arm is controlled to provide the haptic boundary with a second stiffness. The second stiffness is different than the first stiffness, such that process 1300 provides the haptic boundary with different stiffnesses depending on whether the power tool is activated. By repeating the determination of step 1304 throughout execution of a bone modification or other surgical step using the power tool and the haptic boundary, process 1300 can thereby include switching (changing, transitioning, adjusting, etc.) the stiffness of the haptic boundary in response to activation and deactivation of the power tool.

Providing the haptic boundary with a second stiffness in step 1308 can include using a different stiffness parameter in generating force feedback provided by the robotic arm than as used in step 1306. For example, where k₁ is the first stiffness parameter used in step 1306, k₂ can be used as a second stiffness parameter in step 1308 in place of k₁ (k₁≠k₂) in formulations such as those above (e.g., to provide a force feedback based on F=k₂ (1/x), F=(1/x)^{k}^{₂}, or other function of the stiffness parameter). In some embodiments, process 1300 transitions gradually from providing the first stiffness to providing the second stiffness as the power tool is activated or deactivated, for example by providing the stiffness parameter k as a function of time responsive to a user input requesting activation or deactivation of the power tool.

In some embodiments, the second stiffness is lower than the first stiffness, such that the user experiences a softer (e.g., more deformable) haptic boundary when the power tool is activated as compared to when the power tool is inactivated. In other embodiments, the second stiffness is greater than the first stiffness, such that the user experiences the haptic boundary as harder (more rigid, less deformable) when the power tool is activated as compared to when the power tool is inactivated. In some embodiments, the first stiffness and the second stiffness can be set as user (e.g., surgeon) preferences, such that dynamic adjustment of the stiffness of the haptic boundary in process 1300 advantageously provides user-desired performance.

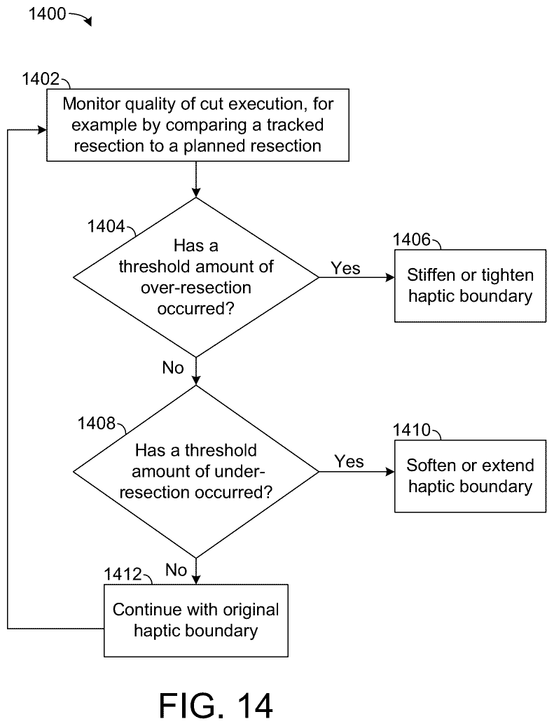

Referring now to FIG. 14, a process for controlling a robotic system is shown, according to some embodiments. Process 1300 can be executed by the computing system 224, for example as part of step 310 of process 300 and/or as part of step 410 of process 400. Process 1300 can be implemented using surgical system 200 and/or robotic device 220 as described above, or other implementations of robotically-assisted surgical systems, in various embodiments. Process 1300 can be provided in combination with process 600, process 900, process 1300, or any of the various teachings provided herein. Process 1400 provides for dynamic modification of the stiffness and/or size of the haptic boundary based on monitored quality of cut execution to adjust for variability in surgeon performance and/or mechanical dynamics which vary across patients, for example to soften or extend a boundary where monitoring shows difficulty in cut completion and stiffening or tightening a boundary where monitoring shows over-resection.

At step 1402, a quality of execution of a cut is monitored. The cut can be a bone resection, for example a bone resection executed using surgical device 234 of surgical system 200 in FIG. 2 and guided by constraining the surgical device 234 to a haptic boundary. The quality of the cut can be monitored as the cut is performed, for example by comparing an accumulation of tracked poses of the surgical device 234 (which can correspond to actual cutting performed) to a planned cut. Such a comparison can be performed using a constructive solid geometry operation using a three-dimensional model of a bone, a three-dimensional model of a planned resection (e.g., of a portion of the bone planned for removal), and a three-dimensional model of accumulated poses of the surgical device relative to the bone as the bone executes a resection (e.g., generated using a tracking system 222 for tracking a bone and the surgical device relative to the bone, described above). Monitoring the quality of execution of the cut can also include timing duration of execution of the cut, i.e., determining an amount of time that has elapsed since the surgical device 234 started to resect the bone (e.g., by starting a timer upon first intersection of a tracked position of the surgical device with the model of the bone).

At step 1404, a determination is made as to whether a threshold amount of over-resection has occurred. Step 1404 can include assessing an amount of a tracked cut which goes beyond the planned cut. A model of accumulated tracked poses of the surgical device can be compared to a model of the bone being cut and a model of a planned resection to determine a region in which the model of accumulated tracked poses overlaps the model of the bone but not the model of the planned resection. Such a region corresponds to over-resection of the bone. Step 1404 can include assessing a size (e.g., volume) of the over-resection region and comparing the size of the over-resection region to a threshold value (e.g., a threshold volume, a threshold percentage of a volume of the model of the planned resection, etc.), and determining that a threshold amount of over-resection amount has occurred when the size of the over-resection region exceeds the threshold value. In some embodiments, a user is prompted to provide input confirming or otherwise indicating that the over-resection has occurred.

In response to occurrence of the threshold amount of over-resection (“Yes” at step 1404), a haptic boundary is stiffened or tightened at step 1406. Step 1406 can include stiffening the haptic boundary, for example by increasing a stiffness parameter used in determining force feedback to be generated by a robotic arm as the surgical device interacts with the haptic boundary (e.g., according to formulas such as those provided elsewhere herein). Stiffening the haptic boundary can make the boundary harder, less pliable, less deformable, etc., providing additional force which resists further over-resection to a greater degree than before execution of step 1406. Step 1406 can additionally or alternatively include tightening the haptic boundary, i.e., moving the perimeter of the haptic boundary inward so as to reduce the interior size of the haptic boundary. In some embodiments, a user input (e.g., surgeon preference, knob adjustment, graphical user interface selection) is used to determine the amount of tightening or stiffening to be provided in step 1406. Providing force feedback based on the tightened haptic boundary renders movement of the surgical device to a position which would correspond to over-resection more difficult (but may also make it more difficult for the surgeon to effectively complete the entire planned resection).

If the threshold amount of over-resection has not occurred (“No” in step 1408), process 1400 proceeds to step 1408 where a determination is made as to whether a threshold amount of under-resection occurred. Step 1408 can include assessing a remaining amount of the planned resection, for example by determining a region of the planned resection which is not overlapped by an accumulation of tracked poses of the cutting tool. A size (e.g., volume) of such not-yet-resection region (remainder of the planned resection) can be compared to one or more criteria in step 1408. For example, in some embodiments the size of said region can be compared to a threshold that decreases as a function of cut duration, i.e., based on an amount of time since the cutting device started resecting the bone, such that a threshold amount of under-resection is determined as having occurred when the size of the remainder of the planned resection exceeds the threshold). As another example, step 1408 is executed at a set amount of time after the cut is initiated, at which time the size of the remainder of the planned resection is compared to a static threshold. As yet another example, a machine learning or other approach can be used to automatically classify a resection as having difficulty reaching a particular region of a planned resection (e.g., in response to a trajectory of the surgical device repeatedly stopping short of reaching such region) to determine the threshold amount of under-resection in step 1408. Step 1408 can thereby correspond to determining whether the surgeon is having difficulty in moving the surgical device through a sufficient range of motion to complete a planned resection. In some embodiments, a user is prompted to provide input confirming or otherwise indicating that the under-resection has occurred.

If a threshold amount of under-resection has occurred (“Yes”) at step 1408 (indicating that the surgeon is having difficulty in moving the surgical device through a sufficient range of motion to complete a planned resection), the haptic boundary is softened or extended at step 1410. Softening the haptic boundary can include reducing a stiffness of the haptic boundary, thereby providing deformability, reduced force feedback, etc. at the haptic boundary which can facilitate the surgeon in pushing against the haptic boundary and move the surgical device to positions which reach the remainder of the planned resection to facilitate completion. Such softening can be achieved by reducing a stiffness parameter used in calculating the amount of force feedback to be generated, as described elsewhere herein.

Step 1410 can include extending the haptic boundary in addition or as an alternative to softening the haptic boundary. Extending the haptic boundary refers to increasing one or more dimensions of the haptic boundary, e.g., moving the haptic boundary outwards from its original position, thereby increasing an available range of motion of the surgical device. Extending the haptic boundary can facilitate the surgeon in moving the surgical device as needed to complete the planned resection. In some embodiments, a user input (e.g., surgeon preference, knob adjustment, graphical user interface selection) is used to determine the amount of extension or softening to be provided in step 1410.

The threshold amount of under-resection has not occurred (“No” at step 1408), process 1400 proceeds to step 1412 where the surgical system continues to operate using the original haptic boundary (e.g., original stiffness, not extended or tightened). Haptic feedback is provided using the original haptic boundary as the surgeon manipulates the surgical device to complete the planned resection. Process 1400 can continue to operate, with monitoring of the quality of cut execution continuing such that the haptic boundary can be stiffened, tightened, softened, or extended according to determination of over- or under-resection according to the steps of process 1400 as the cut is executed. Advantageously, process 1400 can thereby automatically adjust a haptic boundary based on monitor cut performance to facilitate timely and accurate completion of a planned resection.

### Configuration of Exemplary Embodiments

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, magnetic, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations, for example non-transitory computer-readable media. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

What is claimed is:

1. A method of operating a robot of a surgical system, comprising:
monitoring positions of a first interaction point defined relative to a surgical instrument and a second interaction point defined relative to the surgical instrument;
determining a first force feedback based on a first interaction between the first interaction and a boundary based on a first stiffness;
determining a second force feedback based on a second interaction between the second interaction point and the boundary based on a second stiffness; and
controlling the robot to provide a combined force feedback based on the first force feedback and the second force feedback.

2. The method of claim 1, wherein the first interaction point is positioned at a cutting tip of the surgical instrument and the second interaction point is positioned at a shaft of the surgical instrument.

3. The method of claim 1, wherein the boundary comprises a first boundary portion configured to interact with the first interaction point and a second boundary portion configured to interact with the second interaction point.

4. The method of claim 1, wherein:
determining the second force feedback based on the second interaction between the second interaction point and the boundary based on the second stiffness is performed prior to the first interaction point crossing a planned position; and
the method further comprises changing, in response to the first interaction point crossing the planned position, a determination of the second force feedback from being based on the second interaction point interacting with the boundary to being based on the second interaction point interacting with an additional boundary.

5. The method of claim 4, wherein the additional boundary allows a larger range of motion for the second interaction point than the boundary.

6. The method of claim 1, further comprising adjusting, in response to activation of a powered cutting tool of the surgical instrument, the first stiffness.

7. The method of claim 1, further comprising increasing the first stiffness responsive to occurrence of a threshold amount of over-resection.

8. The method of claim 1, further comprising decreasing the first stiffness responsive to occurrence of a threshold amount of under-resection.

9. A method of operating a robot of a surgical system, comprising:
monitoring positions of a first interaction point defined relative to a surgical instrument and as second interaction point defined relative to the surgical instrument;
determining a first force feedback based on a first interaction between the first interaction and a first boundary based on a first stiffness;
determining a second force feedback based on a second interaction between the second interaction point and a second boundary based on a second stiffness; and
controlling the robot to provide a combined force feedback based on the first force feedback and the second force feedback.

10. The method of claim 9, wherein the first interaction point is positioned at a cutting tip of the surgical instrument and the second interaction point is positioned at a shaft of the surgical instrument.

11. The method of claim 9, wherein the second boundary has a different shape than the first boundary.

12. The method of claim 9, further comprising changing the second boundary in response to the first interaction point crossing a planned position.

13. The method of claim 12, wherein changing the second boundary comprises increasing an allowable range of motion for the second interaction point.

14. The method of claim 11, further comprising adjusting, in response to activation of a powered cutting tool of the surgical instrument, the first stiffness.

15. The method of claim 11, further comprising increasing the first stiffness responsive to occurrence of a threshold amount of over-resection.

16. The method of claim 11, further comprising decreasing the first stiffness responsive to occurrence of a threshold amount of under-resection.

17. The method of claim 16, further comprising detecting the threshold amount of under-resection based on a duration of execution of a planned resection.

18. A method of operating a robot of a surgical system, comprising:
monitoring a position of a first interaction point defined relative to a surgical instrument;
controlling the robot to provide a force feedback on the surgical instrument based on interaction between the first interaction point and a haptic boundary, wherein the force feedback is based on a stiffness of the haptic boundary; and
reducing the stiffness of the haptic boundary in response to occurrence of a threshold amount of under-resection relative to a planned resection.

19. The method of claim 18, further comprising increasing the stiffness in response to occurrence of a threshold amount of over-resection relative to the planned resection.

20. The method of claim 18, further comprising determining an additional force feedback based on an additional interaction between a second interaction point and the haptic boundary and controlling the robot to provide a combined force feedback based on the additional force feedback and the force feedback.
